(19) United States
(12) Patent Application Publication
(10) Pub. No.: US 2026/0240666 A1
(43) Pub. Date: Aug. 20, 2026

(54) IMPROVED MECHANICAL JOINTS THAT INCLUDE A COMPACT, BYPASSABLE, SINGLE-SPRING OR NESTED-SPRING WRAP-SPRING CLUTCH

(71) Applicant: Paul Pomeroy, Auburn, WA (US)

(72) Inventor: Paul Pomeroy, Auburn, WA (US)

(21) Appl. No.: 19/474,710

(22) PCT Filed: Apr. 9, 2024
(86) PCT No.: PCT/US2024/023719

§ 371 Date: Oct. 11, 2025

Related U.S. Application Data
(60) Provisional application No. 63/458,864, filed on Apr. 12, 2023.

(51) Int. Cl. A61F 2/64; A61F 2/50; A61F 2/68

(52) U.S. Cl. CPC A61F 2/64; A61F 2002/5073; A61F 2002/6845

(57) ABSTRACT

The current document is directed to improved mechanical joints that each includes a compact, bypassable, single-spring or nested-spring wrap-spring clutch (“BSorNWS clutch”) and that can be incorporated into many different types of devices, appliances, and systems, including prosthetics. One application of a BSorNWS-clutch mechanical joint is as a mechanical knee within a prosthetic leg. A BSorNWS-clutch mechanical-knee component used in a prosthetic leg further includes upper-leg and lower-leg attachments that freely rotate when the prosthetic leg is unweighted but that are constrained, by weighting-induced activation of the BSorNWS-clutch, to rotate only in a lower-leg-extension direction. The BSorNWS-clutch mechanical-knee includes additional components that adjust the weighting force needed to bypass the BSorNWS clutch and to slightly relax the rotational constraint imposed by BSorNWS-clutch activation.

### CROSS-REFERENCE TO RELATED APPLICATION

This application is a 371 of international PCT/US2024/023719, filed Apr. 9, 2024, which claims the benefit of Provisional Application No. 63/458,864, filed Apr. 12, 2023.

### TECHNICAL FIELD

The current document is directed to mechanical joints and, in particular, to improved mechanical joints that each incorporates a compact, bypassable, single-spring or nested-spring wrap-spring clutch.

### BACKGROUND

A mechanical joint connects two or more members and generally provides for one or more types or modes of relative motion of the connected members. For example, a human knee is a complex biomechanical joint that connects an upper leg to a lower leg and that provides for constrained rotation of upper and lower legs with respect to one another. The human knee permits rotation of the lower leg with respect to the upper leg through an angle of nearly 180° in a vertical plane passing through the lower leg, knee, and upper leg that is approximately parallel to the sagittal plane and approximately perpendicular to the coronal plane, but also permits limited rotation of the lower leg within a solid angle extending from the knee when the lower leg is perpendicular to the upper leg as well as small amounts of expansion and compression of the leg. The human body includes many additional types of biomechanical joints, including multiple joints in each finger and toe, ball-and-socket-like joints in hips and shoulders, and elbow, wrist, and ankle joints.

Mechanical joints are used in prosthetics, robots, many different types of machines, and many different types of mechanical and electro-mechanical devices, appliances, and systems. Mechanical joints are designed to provide many different types of relative motions among the members connected by the mechanical joints and the relative motions may be variously constrained and controlled during operation of devices and systems in which the mechanical joints are incorporated. Mechanical joints may be controlled by external control devices and systems, by internal components, and by combinations of external control devices and systems and internal components. Control devices and systems may be purely mechanical, including various types of guides and limiters, may include mechanical, hydraulic, pneumatic, and electro-mechanical actuators, and often include processor-and-memory-implemented control logic.

There are many different trade-offs and competing considerations involved in the design of mechanical joints and the control of mechanical joints. Simple mechanical control of mechanical joints may often be cost-effective and robust, but may not provide complex operational modes and features needed in particular applications. By contrast, complex control devices and systems that incorporate processors and memories along with various types of actuators may allow for arbitrarily complex control that implements complex operational modes and features, but may be expensive to design and implement and may be associated with a large number of failure modes and operational constraints, including the need for providing power to control devices and systems and to various types of actuators.

The design and implementation of a mechanical joint or a particular application of a mechanical joint generally involves an optimization process that balances a variety of trade-offs and competing considerations in order to produce a cost-effective mechanical joint that exhibits desired operational modes and features with desired reliability and robustness. As one example, a variety of different types of mechanical knee joints used in prosthetic legs for amputees have been designed and implemented. Purely mechanical knee joints can be relatively inexpensive, but may exhibit only a few limited operational modes, with users needing extensive training to learn to walk using prosthetic legs that incorporate the purely mechanical knee joints. While purely mechanical knee joints may also be relatively robust, they may lack sufficient control flexibility to provide natural operational modes and thus may not provide users of prosthetic legs that incorporate purely mechanical knee joints with anything close to the safety and ease of use provided by natural legs. By contrast, complex processor-controlled electromechanical knee joints may more accurately simulate the operational modes of natural knees, but may be very expensive to design and produce and may also be limited by the need for external power supplies. The design, implementation, and production of prosthetic legs and other jointed prosthetics thus involves a complex optimization process. Designers, vendors, and users of prosthetic legs and other types of prosthetics continue to seek improved prosthetics that incorporate improved mechanical knees and other improved mechanical joints that can facilitate optimization processes that lower development and production costs while, at the same time, increasing reliability and robustness and providing the operational modes and control flexibility to produce prosthetics that more closely simulate natural limbs and other jointed anatomical features. In similar fashion, designers, vendors, and users of a wide variety of different types of devices, appliances, and systems that incorporate mechanical joints continue to seek improved mechanical joints to facilitate improved optimization of the design and production of many different types of devices, appliances, and systems.

### SUMMARY

The current document is directed to improved mechanical joints that each includes a compact, bypassable, single-spring or nested-spring wrap-spring clutch (“BSorNWS clutch”) and that can be incorporated into many different types of devices, appliances, and systems, including prosthetics. One application of a BSorNWS-clutch mechanical joint is as a mechanical knee within a prosthetic leg. A BSorNWS-clutch mechanical-knee component used in a prosthetic leg further includes upper-leg and lower-leg attachments that freely rotate when the prosthetic leg is unweighted but that are constrained, by weighting-induced activation of the BSorNWS-clutch, to rotate only in a lower-leg-extension direction. The BSorNWS-clutch mechanical-knee includes additional components that adjust the weighting force needed to bypass the BSorNWS clutch and to slightly relax the rotational constraint imposed by BSorNWS-clutch activation.

### BRIEF DESCRIPTION OF THE DRAWINGS

FIGS. 1A-C illustrate characteristics of the human gait cycle.

FIG. 2 illustrates a first problem that would be encountered were a passive rotatable mechanical joint used as a mechanical knee in a mechanical leg prosthesis.

FIG. 3 illustrates a similar problem that would be encountered were a passive rotatable linkage used as a mechanical knee in a mechanical leg prosthesis, using the same illustration conventions as used in FIG. 2.

FIGS. 4A-B show the basic components of a simple, conceptual BSorNWS-clutch mechanical joint from two different perspectives.

FIG. 5 illustrates the BSorNWS-clutch mechanical joint assembled from the BSorNWS-clutch-mechanical-joint components discussed above with reference to FIG. 4A-B.

FIG. 6 shows two different illustrations of the BSorNWS-clutch mechanical joint, assembled from the BSorNWS-clutch-mechanical-joint components discussed above with reference to FIGS. 4A-B, when the BSorNWS is deactivated, or bypassed, due to unweighting of the BSorNWS-clutch mechanical joint.

FIGS. 7A-E show representations of the simple, conceptual BSorNWS-clutch mechanical joint, used as a mechanical knee in a prosthetic leg, at various different positions and time points of the gait cycle illustrated in FIG. 1A.

FIG. 8 shows an exploded diagram of the WSC used in the WSC mechanical joint.

FIG. 9 shows the WSC mechanical joint from a perspective view.

FIGS. 10A-B show one implementation of an inverted single-spring wrap spring (“inverted SSWS”).

FIGS. 11A-D illustrate one implementation of the currently disclosed BSorNWS-clutch mechanical joint that is incorporated into a BSorNWS-clutch prosthetic leg.

FIG. 12 illustrates this alternative bypass mechanism.

FIG. 13 illustrates one implementation of a flexion-prevention-override mechanism that allows a small amount of controlled flexion of the BSorNWS-clutch mechanical joint.

FIG. 14 illustrates another improvement incorporated into the currently disclosed BSorNWS-clutch mechanical joint.

FIG. 15 illustrates a nested-spring wrap spring (“NSWS”) and a BSorNWS clutch containing an NSWS.

FIGS. 16A-C illustrate an alternative flexion-prevention-override mechanism that allows a small amount of rotation of the lower-leg attachment with respect to the upper-leg attachment in the flexion direction when the BSorNWS-clutch mechanical joint is weighted.

FIG. 17 shows an alternative implementation of the lower-leg attachment that, as discussed above, translates with respect to the housing depending on whether or not the lower-leg attachment is weighted or unweighted.

FIGS. 18A-C illustrate yet a different, alternative implementation of the lower-leg attachment.

FIGS. 19A-B illustrate an alternative bypass mechanism.

### DETAILED DESCRIPTION

The current document is directed to mechanical joints that each incorporates a bypassable single-spring or nested-spring wrap-spring clutch (“BSorNWS clutch”) and to the use of a BSorNWS-clutch-incorporating mechanical joint as a mechanical knee within a prosthetic leg. In a first subsection, below, the human gait cycle is discussed. In a second subsection, desirable mechanical-knee operational characteristics are discussed. In a third subsection, a simple, conceptual BSorNWS-clutch mechanical joint is discussed and illustrated to clearly describe the features and operational characteristics of the currently disclosed BSorNWS-clutch-incorporating mechanical joints. In a fourth subsection, one implementation of the currently disclosed BSorNWS-clutch-incorporating mechanical joints is discussed. In a final subsection, various alternative implementations of the currently disclosed BSorNWS-clutch-incorporating mechanical joints are discussed.

### Gait Cycle

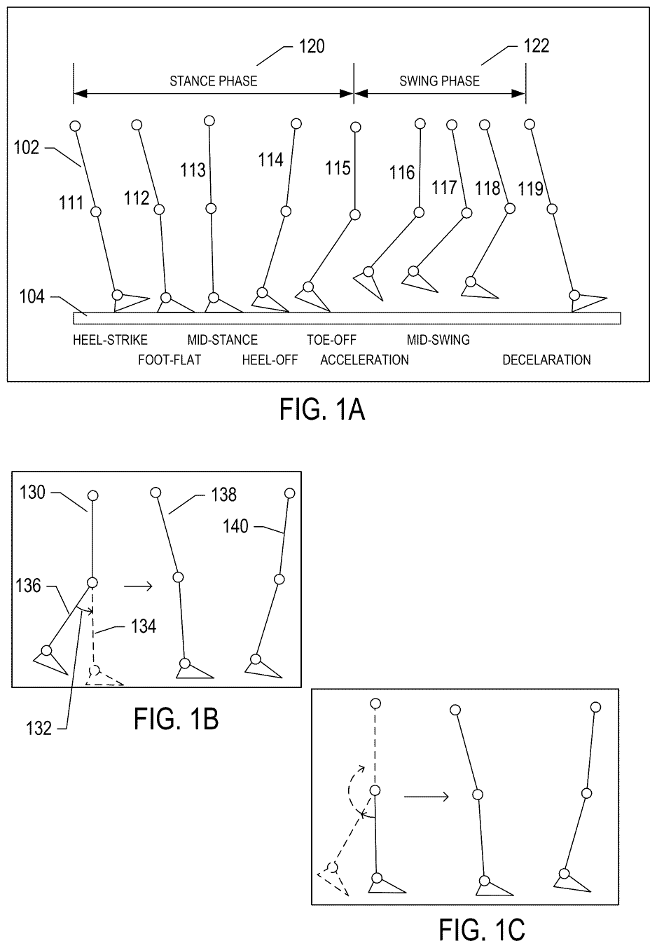

FIGS. 1A-C illustrate characteristics of the human gait cycle. FIG. 1A illustrates the human gait cycle. FIG. 1A shows a stick-figure representation of a leg, including a first representation 102, at various points in time within the gait cycle and at various positions along a horizontal surface 104. The gait cycle begins at a first position and time point 111 and ends at a final position and time point 118 immediately preceding the beginning of the next gait cycle at position and time point 119. Assuming that the representation of the leg in FIG. 1A is a representation of the left leg of a user, the user's right leg would follow the same gait cycle, but the gait cycle of the right leg would be displaced, in time, relative to the gait cycle of the left leg. For example, when the left leg is at position and time point 111, at the beginning of the left leg's gait cycle, the right leg would be at position and time point 114, halfway through the right leg's gait cycle.

At position and time point 111, the user's left leg has been extended outward in the direction of walking and the heel of the user's left foot placed forward of the user's center of mass onto the horizontal surface. Up until the heel of the user's left foot contacts the horizontal surface, the user's weight is entirely supported by the right leg, which is bent to allow the user's center of mass to advance to a position forward from the position of the toes of the user's right foot. Then, in the sequence of positions and time points 112-114, the user's center of mass proceeds in a forward direction with the user's weight transferred to the left leg as the user's right leg bends further in order to allow the right leg to be disengaged from the horizontal surface and swung forward, as in the sequence of positions and time points 116-119. Thus, the gait cycle for each leg is divided into a stance phase 120 and a swing phase 122. During the stance phase, the user's weight is fully transferred to the leg and the user's center of mass, initially behind the leg, is propelled forward to a position ahead of the leg in order that the other leg can be lifted and swung forward during the swing phase. During the swing phase, the lower leg rotates counterclockwise with respect to the upper leg, in the orientation depicted in FIG. 1A, in order for the lower leg to be extended forward, in the direction of walking. This counterclockwise rotation is referred to as “extension.” During the stance phase, the lower leg rotates in a clockwise direction with respect to the upper leg, in the orientation depicted in FIG. 1A, to allow the user's center of mass to advance from behind the leg to a position forward of the leg. This clockwise rotation is referred to as “flexion.”

FIGS. 1B-C illustrate extension and flexion. In FIG. 1B, the stick-figure representation of a leg 130 shows the lower leg bent backwards with respect to the upper leg. The direction of walking is from left to right, as in FIG. 1A. Curved arrow 132 and the dashed lower leg 134 represent a counterclockwise rotation of the lower leg with respect to the upper leg. The final position of the lower leg following the counterclockwise rotation is represented by the dashed-line lower leg 134. This counterclockwise rotation, as mentioned above, represents extension of the lower leg with respect to the upper leg. The knee permits extension up to the point that the lower and upper legs are parallel and forming an approximately straight-line segment through the knee. Extension of the solid-line lower leg 136 to the position of the dashed-line lower leg 134 along with a change in the orientation of the upper leg 130 can produce many different resulting leg positions and orientations, including leg positions and orientations 138 and 140 shown in FIG. 1B. Of course, were the leg viewed from the other side, with the walking direction from right to left, then extension of the lower leg with respect to the upper leg would involve a clockwise rotation rather than a counterclockwise rotation. Using the same illustration conventions as used in FIG. 1B, FIG. 1C illustrates flexion of the lower leg with respect to the upper leg. In the orientation shown in FIGS. 1A-C, flexion results from a clockwise rotation of the lower leg with respect to the upper leg. However, were the orientation to change by 180°, with the walking direction from right to left, then flexion would result from a counterclockwise rotation of the lower leg with respect to the upper leg. The transition from position and time point 111 to position and time point 112 in FIG. 1A results from a relatively slight flexion of the lower leg with respect to the upper leg and the transition from position and time point 112 to the position and time point 113 in FIG. 1A represents extension of the lower leg with respect to the upper leg. In the gait cycle, flexion reduces the angle between the upper leg and lower leg and extension increases the angle between the lower leg and upper leg.

### Desirable Mechanical-Knee Operational Characteristics

Transfemoral amputees use prosthetic legs for walking and for other activities. An ideal prosthetic leg would have the same weight and dimensions of a natural leg, would receive any required electrical power or other types of power from internal batteries or other power supplies with infrequent need for recharging, and would be controlled by control inputs from the user's nervous system. Unfortunately, an ideal prosthetic leg is currently neither available nor anticipated to be available in the near future. In fact, the various types of prosthetic knees used in prosthetic legs generally fail to provide both the range of relative motions for the upper-leg and lower-leg members of the prosthetic leg that occur in natural legs as well as the sophisticated control features needed to implement the operational characteristics of natural legs. Currently available prosthetic legs include microprocessor-controlled electromechanical prosthetic legs and mechanical prosthetic legs. Users can often master the use of microprocessor-controlled prosthetic legs more quickly than they can master the use of mechanical prosthetic legs. Microprocessor-controlled prosthetic legs generally provide a broader range of operational modes and operational characteristics than mechanical prosthetic legs and function more similarly to natural legs than mechanical prosthetic legs. Research efforts have even produced microprocessor-controlled prosthetic legs that can be controlled by inputs from a user's nervous system. However, microprocessor-controlled prosthetic legs are generally quite expensive, and therefore unavailable to a large fraction of amputees needing prosthetic legs, are often powered by batteries that have relatively short recharging intervals, and are subject to many different types of failures and malfunctions due to the complexity of their components. By contrast, while not providing the broad range of operational modes and characteristics provided by microprocessor-controlled prosthetic legs, mechanical prosthetic legs are generally much cheaper, often more durable, and easier to manufacture. Note that, in this and following paragraphs, the phrase “mechanical joint” refers to a fully or pure mechanical joint that lacks electronic, pneumatic, or hydraulic control subsystems or devices. The currently disclosed and claimed mechanical joint is a purely or fully mechanical joint that, when incorporated into a prosthetic leg, renders the prosthetic leg far less expensive and generally more robust and reliable than the prosthetic legs that include electronic, electromechanical, hydraulic, and/or mechanical-hydraulic control components and actuators. The currently disclosed and claimed mechanical joint additionally provides a broader range of operational modes and operational characteristics than currently available mechanical prosthetic legs.

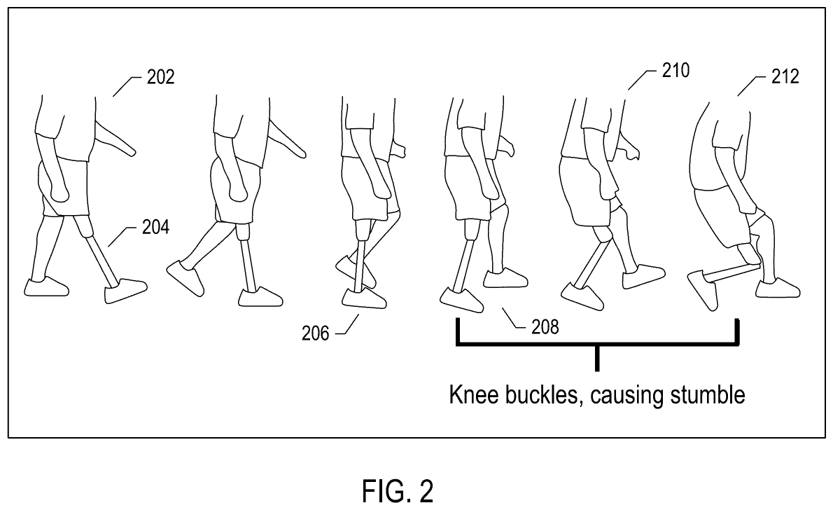

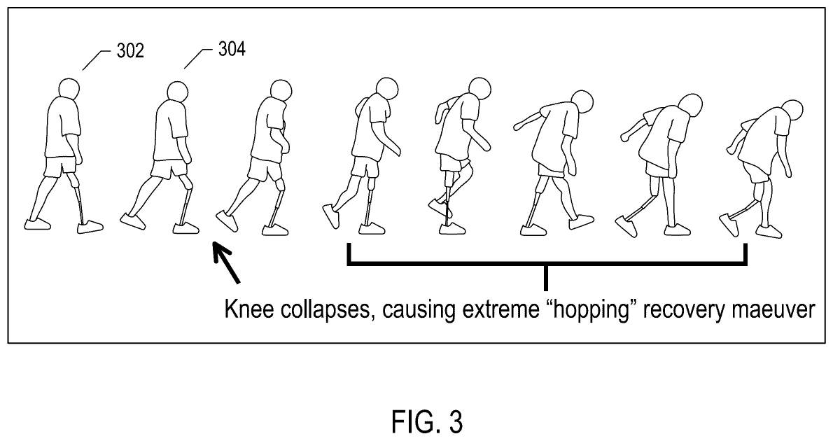

The desired functionalities of a mechanical knee used in a mechanical leg prosthesis can be inferred by considering the gait cycle, discussed above with reference to FIG. 1A. As discussed above, a mechanical knee joins, or connects, an upper-leg member to a lower-leg member and needs to provide for both extension and flexion of the lower-leg member. However, a passive rotatable mechanical joint is insufficient for implementation of a usable prosthetic leg. FIG. 2 illustrates a first problem that would be encountered were a passive rotatable mechanical joint used as a mechanical knee in a mechanical leg prosthesis. FIG. 2 shows a sequence of representations of a patient 202 with a prosthetic leg 204 walking in the left-to-right direction. In representation 206, the patient's prosthetic leg is at or near the position or time point 113 in FIG. 1A. As the center of mass of the patient continues to move in a forward direction, and the patient begins to lean forward in order to swing the patient's left leg forward, as shown in representation 208, which corresponds to the position or time point 114 in FIG. 1A, a passive rotatable mechanical joint would begin to fail and then buckle, as illustrated in representations 210 and 212 in FIG. 2. Thus, while flexion is needed during the swing phase of the gait cycle or a prosthetic leg, flexion during the stance phase needs to be prevented in order to avoid buckling of the prosthetic leg and a resulting stumble or fall. FIG. 3 illustrates a similar problem that would be encountered were a passive rotatable linkage used as a mechanical knee in a mechanical leg prosthesis, using the same illustration conventions as used in FIG. 2. This problem occurs at an earlier time point in the gait cycle than the problem illustrated in FIG. 2, when the prosthetic leg is at the position or time point 112 in the gait cycle shown in FIG. 1A, as shown in representations 302 and 204 in FIG. 3. The user has not fully transferred his or her weight to the prosthetic leg, but the lower leg has slightly rotated clockwise with respect to the upper leg after full extension at position or time point 111 in FIG. 1A. The prosthetic leg begins to collapse, rather than reextending to reach position or time point 113. Therefore, at minimum, a mechanical knee joint should prevent substantial flexion of the lower leg with respect to the upper leg at positions or time points 112 and 114, allow a small amount of controlled flexion at positions or time points 111 and 113, and allow substantial flexion at positions or time points 115-117. The mechanical knee joint should allow for free extension of the lower leg with respect to the upper leg during the swing phase of the gait cycle up to the point that the upper leg and lower leg are approximately parallel, as in positions or time points 111, 113, and 119 in FIG. 1A. In fact, due to the dynamics of the gait cycle, a mechanical knee needs only to ensure that extension cannot continue past the point that the upper leg and lower leg are approximately parallel, but can otherwise permit extension at any time point during the gait cycle.

There are many different possible ways to attempt to mechanically control a mechanical knee joint in order to constrain flexion at appropriate time points and positions during the gait cycle. In an approach used in the currently disclosed mechanical joints that each incorporates a bypassable single-spring or nested-spring wrap-spring clutch (“BSorNWS clutch”), referred to as (“BSorNWS-clutch mechanical joints”) in the discussion below, control of flexion constraint during the gait cycle is based on whether or not the prosthetic leg containing the BSorNWS-clutch mechanical joint, referred to as a (“BSorNWS-clutch prosthetic leg”) in the discussion below, is bearing more than a threshold percentage or portion of the patient's weight. During most of the swing phase of the gait cycle, as discussed above, the lower-leg member of the prosthetic leg is unweighted, with the foot of the prosthetic leg suspended above the walking surface. This occurs during positions and time points 116-118 in FIG. 1A. Flexion need not be constrained during this portion of the gait cycle and, in fact, needs to be permitted by the mechanical knee joint to facilitate suspension of the foot above the walking surface. By contrast, during the stance phase of the gait cycle following position and time point 112 and preceding position and time point 114, flexion needs to be fully constrained in order to prevent buckling of the prosthetic leg that leads to stumbles and falls, as discussed above. During this portion of the stance phase of the gait cycle, the prosthetic leg is bearing all or a large portion of the user's weight. Thus, by detecting when the prosthetic leg is bearing more than a threshold percentage or portion of the user's weight, the mechanical control feature or features of a mechanical knee joint can constrain flexion for the appropriate portion of the stance phase of the gait cycle and can permit flexion for the appropriate portion of the swing phase of the gait cycle.

There are a few additional, more subtle operational characteristics that a mechanical knee needs to exhibit. First, the mechanical knee should not exhibit sharp mode or state transitions so that, for example, even as flexion is generally being constrained during the stance phase of the gait cycle, a small amount of flexion is nonetheless allowed. For example, as discussed above, as the heel of the prosthetic leg contacts the horizontal surface, in position or time point 111, a small amount of flexion needs to be permitted as the prosthetic leg transitions through position and time point 112 to full weight bearing in position and time point 113. Second, the force applied to control constraint of flexion needs to vary with respect to the relative orientation of the lower and upper legs to facilitate transition from a sitting to a standing position. These additional operational characteristics are provided, in a disclosed implementation, by additional mechanical features of the currently disclosed BSorNWS-clutch mechanical joints that are discussed in later sections of this document.

### Conceptual Illustration of the Currently Disclosed BSorNWS-Clutch Mechanical Joint

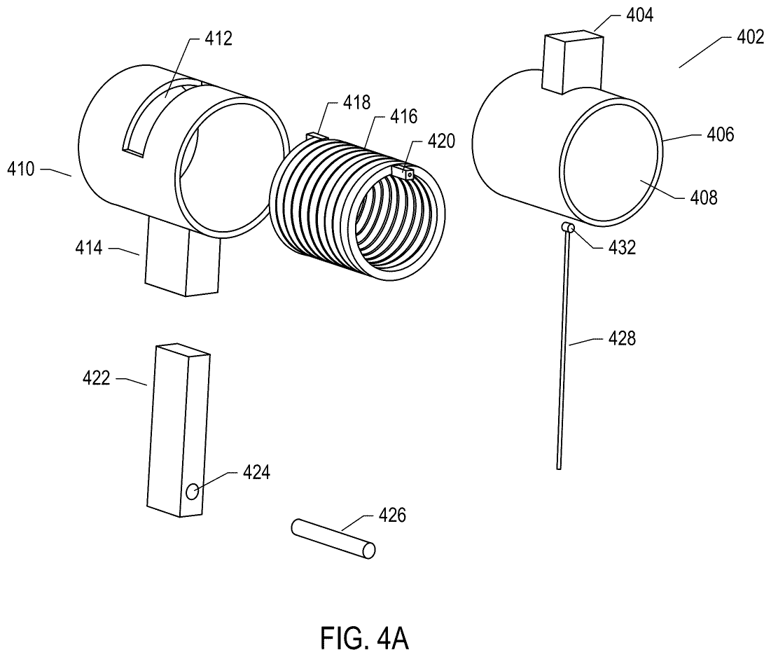

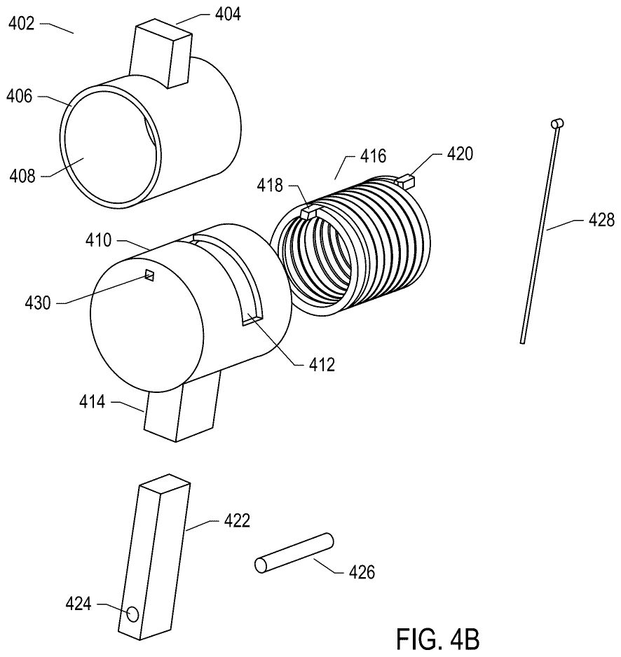

In the current section of this document, a simple, conceptual BSorNWS-clutch mechanical joint is described with reference to FIGS. 4A-7E. The simple, conceptual BSorNWS-clutch mechanical joint is described and illustrated in order to simply and clearly describe basic components of the currently disclosed BSorNWS-clutch mechanical joint and operation of the BSorNWS-clutch mechanical joint at the various positions and time points of the gait cycle discussed above and illustrated in FIG. 1A. Again, the currently disclosed BSorNWS-clutch mechanical joint is a purely or fully mechanical joint. The simple, conceptual BSorNWS-clutch mechanical joint has not been implemented and would likely not be implemented because there is no known application for the simple, conceptual BSorNWS-clutch mechanical joint other than to illustrate basic components and component functionalities of a BSorNWS-clutch mechanical joint and because the simple, conceptual BSorNWS-clutch mechanical joint would likely be impossible or impractical to manufacture. The relative component sizes, component orientations, and other characteristics of the simple, conceptual BSorNWS-clutch mechanical joint are, in certain cases, exaggerated with respect to the actual relative component sizes and component orientations in BSorNWS-clutch mechanical joints implemented for practical applications, including for use as mechanical knees mechanical prosthetic legs.

FIGS. 4A-B show the basic components of the simple, conceptual BSorNWS-clutch mechanical joint from two different perspectives. The basic components include: (1) an upper-leg component 402 comprising an upper-leg attachment shaft 404 mounted to a cylindrical member 406 with an inner cylindrical friction surface 408; (2) a cylindrical housing 410 with a slot 412 to accommodate the upper-leg attachment shaft 404 to which a lower-leg-attachment-shaft fitting 414 is mounted; (3) an inverted single-spring wrap spring (“inverted SSWS”) 416 with two small horizontal attachment features 418 and 420; (4) a lower-leg attachment shaft 422 that this slidably mounted within the lower-leg-attachment-shaft fitting 414 and that includes a bypass-pin hole 424; (5) a bypass pin 426; and (6) a bypass linkage 428. The upper-leg and the lower-leg attachment shafts, 404 and 422, respectively, attach the simple, conceptual BSorNWS-clutch mechanical joint to an upper-leg member and a lower-leg member of a BSorNWS-clutch prosthetic leg. Rotation of the upper-leg component 402 within the cylindrical housing 410 provides extension and flexion to the BSorNWS-clutch mechanical joint and BSorNWS-clutch prosthetic leg. The inverted SSWS 416 implements the BSorNWS-clutch. The wrap-spring-coil attachment feature 418 mounts into aperture 430 within the housing 410 in order to fix the inverted SSWS to the housing. The inverted SSWS resides within the cylindrical member 406 of the upper-leg component 402 and the upper-leg component in turn resides within the cylindrical housing 410, with the upper-leg attachment shaft 404 protruding outward, through slot 412 in the cylindrical housing 410. Expansion of the inverted SSWS outward against the cylindrical friction surface 408 of the cylindrical member 406 of the upper-leg component 402 locks the rotational orientation of the cylindrical member 406 of the upper-leg component 402 with respect to the cylindrical housing 410 when a rotational force is applied that would otherwise result in rotation of the lower-leg attachment shaft in the flexion direction with respect to the upper-leg attachment shaft. The bypass linkage 428 is rotationally attached, via small cylindrical feature 432, to the small attachment feature 420 of the inverted SSWS and is also attached to the bypass pin 426 which is rotationally mounted in the bypass-pinhole 424 towards the distal end of the lower-leg attachment shaft 422 slidably mounted within the lower-leg-attachment-shaft fitting 414. When the BSorNWS-clutch prosthetic leg is unweighted, the lower-leg attachment shaft 422 slides downward, within the lower-leg-attachment-shaft fitting 414, pulling the bypass linkage 428 downward and resulting in a counterclockwise rotation, from the perspective shown in FIG. 4A, of the end of the inverted SSWS which tightens the inverted SSWS and releases the cylindrical member 406 of the upper-leg component 402 to freely rotate within the cylindrical housing 410. When the BSorNWS-clutch prosthetic leg is weighted, the lower-leg attachment shaft 422 slides upward, within the lower-leg-attachment-shaft fitting 414, allowing the inverted SSWS to expand outward to contact the upper-leg component 402 to the cylindrical housing 410. Thus, the lower-leg attachment shaft 422 moves vertically within the lower-leg-attachment-shaft fitting 414 in response to weighting and unweighting of the BSorNWS-clutch prosthetic leg, and this vertical movement of the lower-leg attachment shaft 422 is transduced into an unwinding or winding of the inverted SSWS 416 that activates and deactivates the BSorNWS clutch.

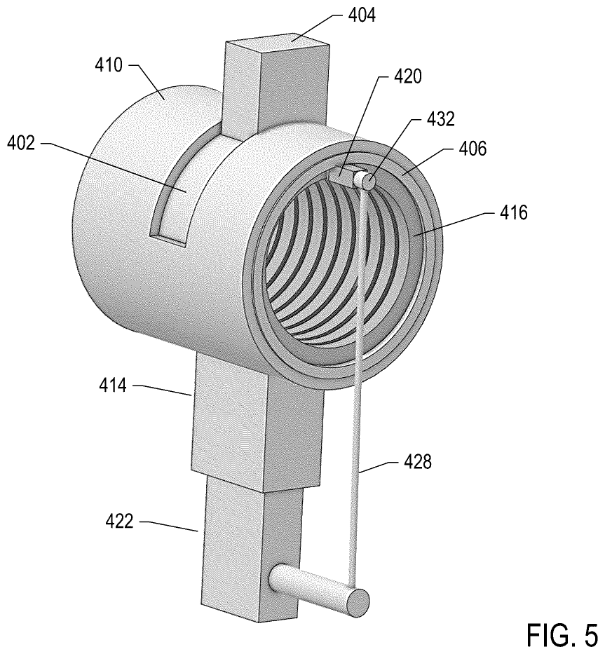

FIG. 5 illustrates the BSorNWS-clutch mechanical joint assembled from the BSorNWS-clutch-mechanical-joint components discussed above with reference to FIGS. 4A-B. In FIG. 5, the BSorNWS clutch is activated and, therefore, rotation of the lower-leg with respect to the upper-leg attachment in the flexion direction is prevented or constrained. The BSorNWS-clutch mechanical joint is weighted, resulting in the lower-leg attachment shaft 422 translated upward into the lower-leg-attachment-shaft fitting 414 which, in turn, moves the bypass linkage upward so that the inverted SSWS is not tightened by the bypass linkage and therefore has expanded so that the outer surfaces of the coils of the inverted SSWS are pushed against the inner, friction surface of the cylindrical member 406 of the upper-leg component 402. As discussed further, below, the lower-leg attachment shaft 422 may include a spring within the lower-leg-attachment-shaft fitting 414 that is compressed when the BSorNWS-clutch mechanical joint is weighted so that, when the weighted BSorNWS-clutch mechanical joint is unweighted, the lower-leg attachment shaft 422 is forced downward by decompression of the spring. The BSorNWS-clutch is activated by weighting of the BSorNWS-clutch mechanical joint regardless of the rotational orientation of the upper-leg component 402 and upper-leg attachment shaft 404 within the cylindrical housing 410.

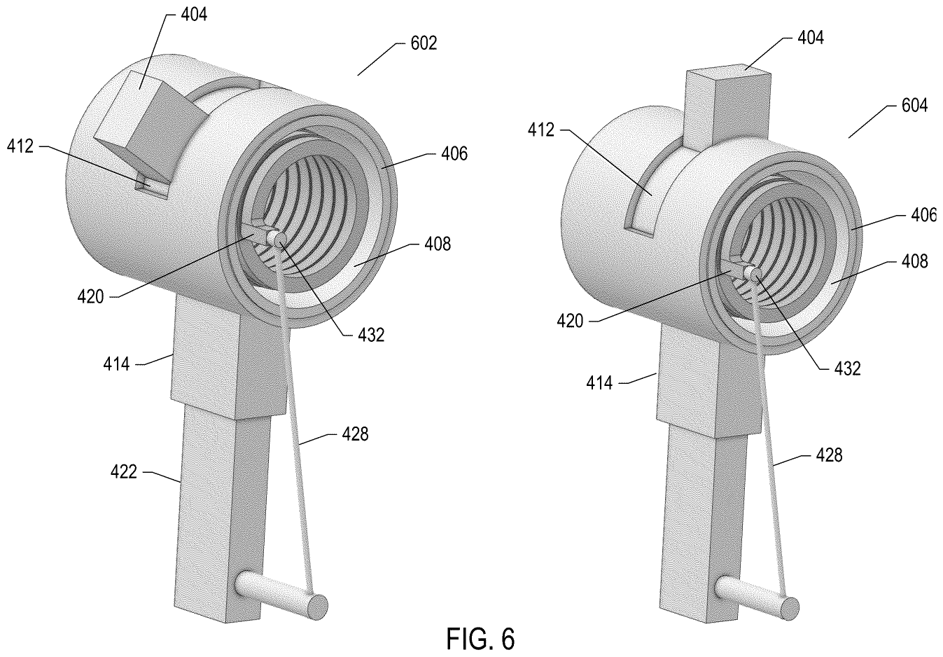

FIG. 6 shows two different illustrations of the BSorNWS-clutch mechanical joint, assembled from the BSorNWS-clutch-mechanical-joint components discussed above with reference to FIGS. 4A-B, when the BSorNWS is deactivated, or bypassed, due to unweighting of the BSorNWS-clutch mechanical joint. In the first illustration 602 of the BSorNWS-clutch mechanical joint, the upper-leg attachment shaft 404 is rotated downward within slot 412 with respect to the lower-leg attachment shaft 422. In the second illustration 604, the upper-leg attachment shaft 404 has been rotated upward within slot 412 with respect to the position of the upper-leg attachment shaft in illustration 602. When the BSorNWS-clutch mechanical joint is incorporated into a BSorNWS-clutch prosthetic leg, the orientation of the upper-leg attachment shaft with respect to the lower-leg attachment shaft in illustration 602 corresponds to a significant flexion of the lower-leg member with respect to the upper-leg member of the BSorNWS-clutch prosthetic leg. By contrast, the orientation of the upper-leg attachment shaft with respect to the lower-leg attachment shaft in illustration 604 corresponds to full extension of the lower-leg member with respect to the upper-leg member of the BSorNWS-clutch prosthetic leg. In both illustrations 602 and 604 in FIG. 6, due to unweighting of the BSorNWS-clutch mechanical joint and decompression of the spring within the lower-leg-attachment-shaft fitting 414 which, together with the force of gravity, forces the lower-leg attachment shaft 422 downward, the extended lower-leg attachment shaft has pulled the bypass linkage 428 downward, tightening the coils of the inverted SSWS so that the outer surfaces of the coils no longer contact the inner, friction surface 408 of the cylindrical member 406 of the upper-leg component, deactivating the BSorNWS clutch and permitting free rotation of the upper-leg attachment shaft with respect to the lower-leg attachment shaft 422 over a range of angles from full flexion to full extension.

It should be noted that the large difference in the effective length of the lower-leg attachment shaft in the activated state of the BSorNWS clutch shown in FIG. 5 with respect to the deactivated state of the BSorNWS clutch shown in FIG. 6, as well as the 90° rotation of the wrap-spring-coil attachment shaft 420 and cylindrical feature 432 from their position in FIG. 5 to their positions in FIG. 6, are exaggerated with respect to the actual degree of lower-leg attachment shaft extension and retraction and movement of the bypass linkage in actual implementations discussed below. As mentioned above, the simple, conceptual BSorNWS-clutch mechanical joint discussed and illustrated in the current section of this current document is used to clearly illustrate operation of the BSorNWS-clutch mechanical joint, when used as a mechanical knee within a mechanical prosthetic leg, during the gait cycle discussed above with reference to FIG. 1A.

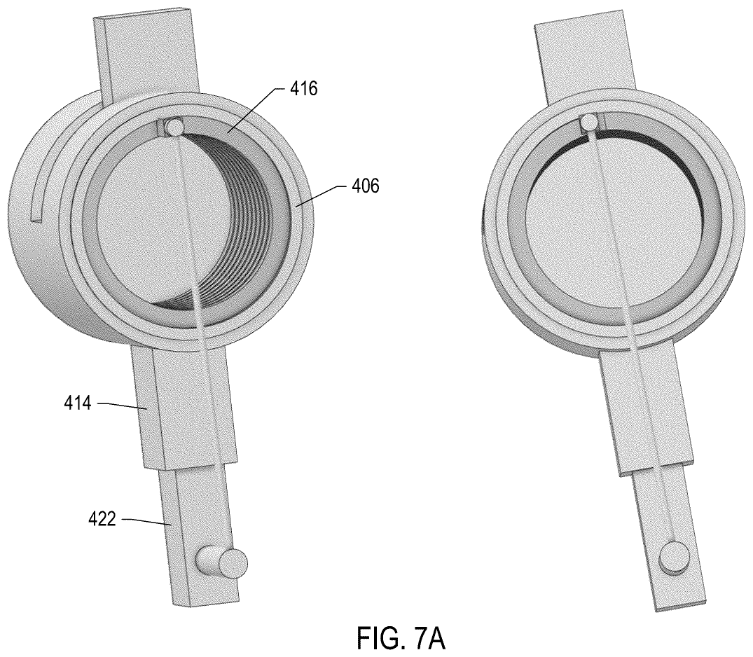

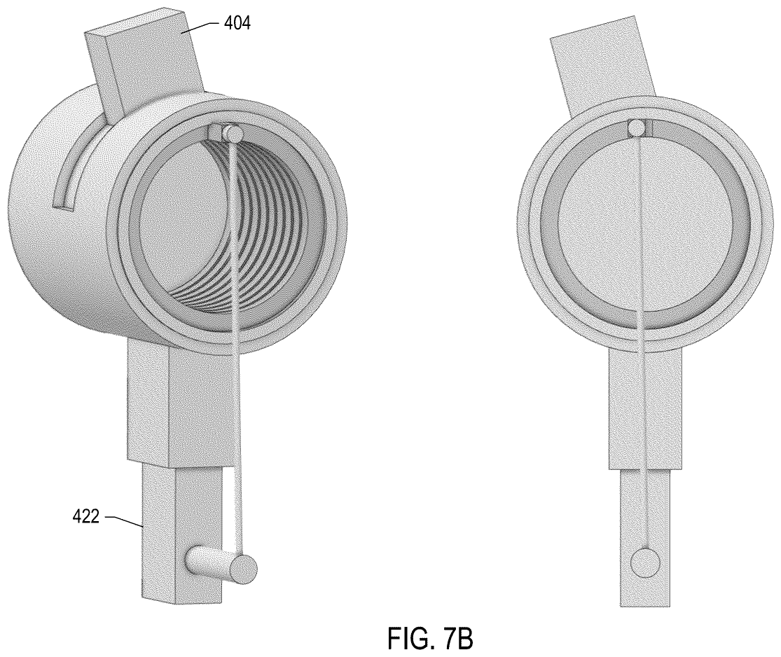

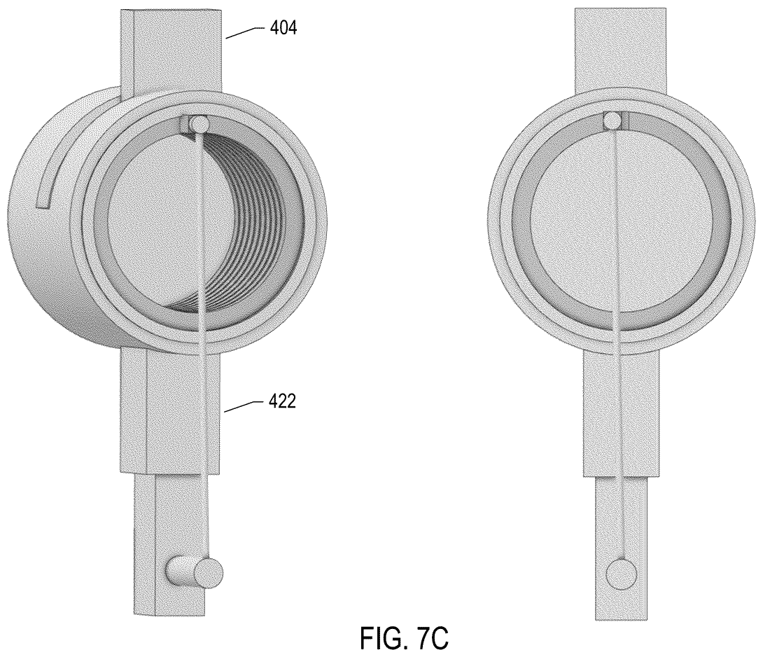

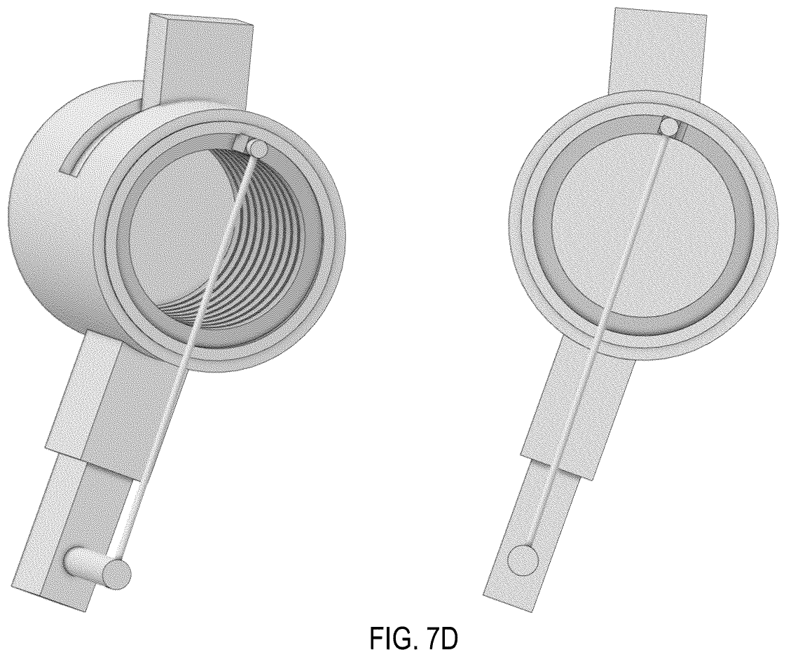

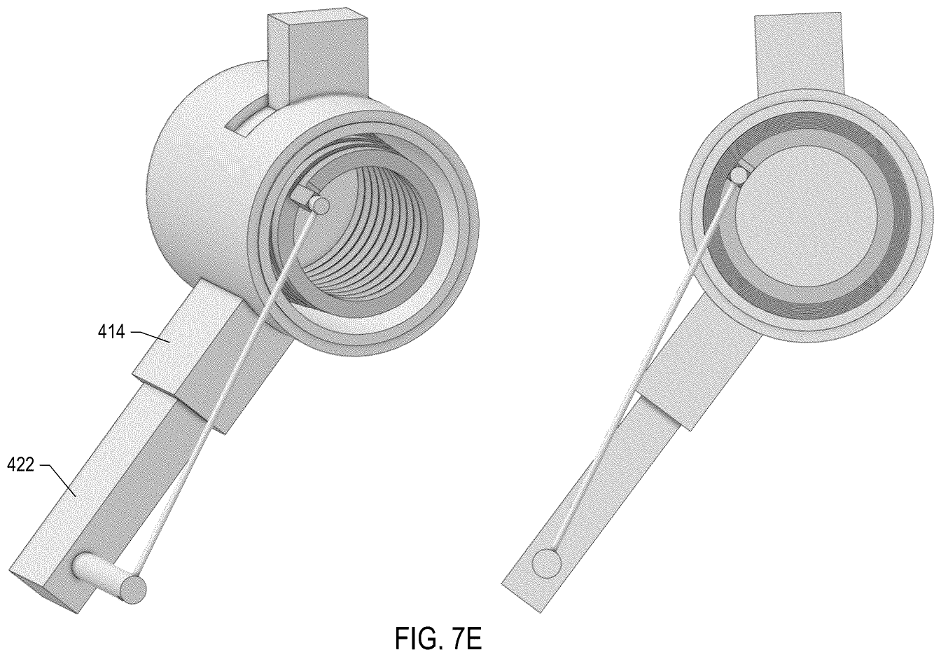

FIGS. 7A-E show representations of the simple, conceptual BSorNWS-clutch mechanical joint, used as a mechanical knee in a prosthetic leg, at various different positions and time points of the gait cycle illustrated in FIG. 1A. Each of FIGS. 7A-E shows the simple, conceptual BSorNWS-clutch mechanical joint from two different perspectives or viewpoints. FIG. 7A shows the simple, conceptual BSorNWS-clutch mechanical joint as it would be oriented and configured at positions and time points 111 and 119 in FIG. 1A. At positions and time points 111 and 119, the prosthetic leg is partially weighted, having just been fully extended and lowered so that the heel of the foot touches the walking surface and the user's weight begins to be transferred to the prosthetic leg. Because the weighting of the prosthetic leg is greater than a threshold level or degree of weighting needed to activate the BSorNWS clutch, the clutch is activated and rotation of the lower-leg attachment with respect to the upper-leg attachment shaft in the flexion direction is constrained. Note that the lower-leg attachment shaft 422 has been pushed into the lower-leg-attachment-shaft fitting 414, compressing the internal spring and allowing the inverted SSWS 460 to expand to press against the internal friction surface of the cylindrical member 406 of the upper-leg component. The long axes of the upper-leg attachment shaft and lower-leg attachment shaft are parallel.

FIG. 7B shows the simple, conceptual BSorNWS-clutch mechanical joint as it would be oriented and configured at position and time point 112 in FIG. 1A. At position and time point 112, the weighting of the prosthetic leg has increased, with the degree of weighting is well above the threshold of weighting needed to deactivate the BSorNWS clutch. As a result, the clutch remains activated and rotation of the lower-leg attachment with respect to the upper-leg attachment in the flexion direction is constrained. However, the long axes of the upper-leg attachment shaft and lower-leg attachment shaft 422 are no longer parallel, as they were in FIG. 7A because, at position and time point 112, a small amount of flexion has taken place, as evident from the non-parallel orientations of the upper-leg and lower-leg attachment shafts. This small amount of flexion, as mentioned above, is permitted by an additional feature of the BSorNWS-clutch mechanical joint that is not included in the simple, conceptual BSorNWS-clutch mechanical joint illustrated in FIGS. 4A-7E but that is discussed below, in a following subsection of this document.

FIG. 7C shows the simple, conceptual BSorNWS-clutch mechanical joint as it would be oriented and configured at position and time point 113 in FIG. 1A. At position and time point 113, the weighting of the prosthetic leg is maximal, with the degree of weighting well above the threshold of weighting needed to deactivate the BSorNWS clutch. As a result, the clutch remains activated and rotation of the lower-leg attachment shaft 422 with respect to the upper-leg attachment shaft 404 in the flexion direction is constrained. The long axes of the upper-leg attachment shaft and lower-leg attachment shaft are again parallel, as they were in FIG. 7A.

FIG. 7D shows the simple, conceptual BSorNWS-clutch mechanical joint as it would be oriented and configured at position and time point 114 in FIG. 1A. At position and time point 114, the weighting of the prosthetic leg is maximal, with the degree of weighting well above the threshold of weighting needed to deactivate the BSorNWS clutch. As a result, the clutch remains activated and rotation of the lower-leg attachment with respect to the upper-leg attachment in the flexion direction is constrained. However, the long axes of the upper-leg attachment shaft and lower-leg attachment shaft are no longer parallel, as they were in FIG. 7C, with a small amount of flexion again exhibited by the BSorNWS-clutch mechanical joint as the user's center of mass has moved forward of the BSorNWS-clutch mechanical joint as the user is preparing to begin to transfer the user's weight to the user's natural leg. The small amount of flexion, as mentioned above, is permitted by an additional feature of the BSorNWS-clutch mechanical joint that is not included in the simple, conceptual BSorNWS-clutch mechanical joint illustrated in FIGS. 4A-7E.

FIG. 7E shows the simple, conceptual BSorNWS-clutch mechanical joint as it would be oriented and configured at position and time point 115 in FIG. 1A. At position and time point 115, the user is preparing to lift the lower-leg member of the prosthetic leg, having transferred most of the user's weight to the user's natural leg. The weighting of the prosthetic leg has fallen below the level or degree of weighting required to activate the BSorNWS clutch. As a result, the BSorNWS clutch has been deactivated and rotation of the lower-leg attachment shaft with respect to the upper-leg attachment shaft is no longer constrained, with the lower-leg attachment shaft and lower-leg member of the prosthetic leg able to freely rotate with respect to the upper-leg attachment shaft and upper-leg member of the prosthetic leg over a range of angles from full flexion to full extension. Note that the lower-leg attachment shaft 422 is now extended outward from the lower-leg-attachment-shaft fitting 414. The BSorNWS clutch of the simple, conceptual BSorNWS-clutch mechanical joint remains deactivated and the lower-leg attachment shaft and lower-leg member of the prosthetic leg able to freely rotate with respect to the upper-leg attachment shaft and upper-leg member of the prosthetic leg at positions and time points 116-118 in FIG. 1A, with relatively minor changes in the degree of flexion as the lower-leg attachment shaft and lower-leg member of the prosthetic leg or swung forward while the lower-leg member of the prosthetic leg is suspended above the walking surface. Finally, in the transition from position and time point 118 in FIG. 1A to position and time point 119 in FIG. 1A, the lower-leg attachment shaft and lower-leg member of the prosthetic leg are swung forward to full extension and the heel of the foot of the lower-leg member is lowered to the walking surface, with sufficient weight then transferred to the lower-leg attachment shaft and lower-leg member of the prosthetic leg to reactivate the BSorNWS clutch, as in FIG. 7A.

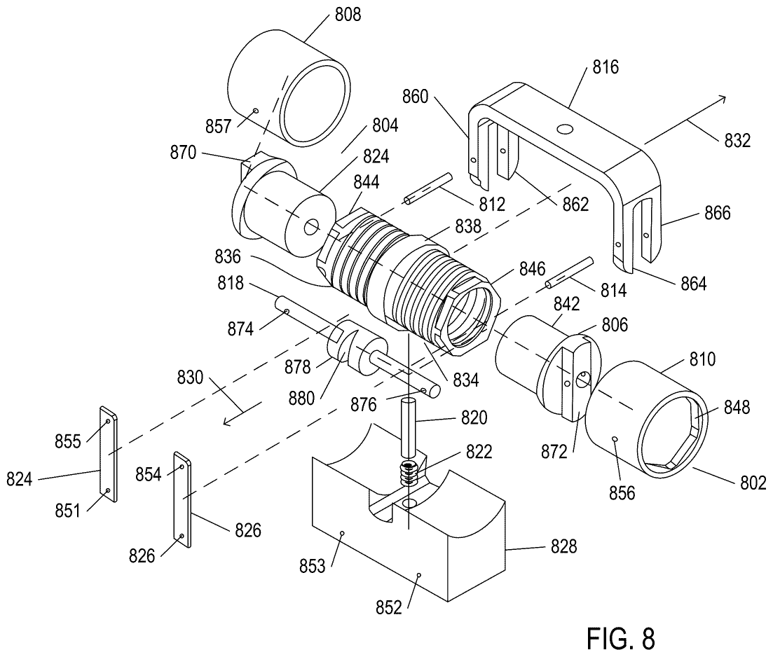

There are a number of significant features and characteristics of the currently disclosed BSorNWS-clutch mechanical joint that are shown in the simple, conceptual BSorNWS-clutch mechanical joint illustrated in FIGS. 4A-7E and that differ from features and characteristics of a dual-wrap-spring-clutch (“WSC”) mechanical joint that was previously disclosed in U.S. Pat. No. 11,020,247. FIG. 8 shows an exploded diagram of the WSC used in the WSC mechanical joint. The WSC 802 is based on a dual wrap spring comprising a central band 838 from which two outward-spiraling helical coils 834 and 836 extend. By contrast, the currently disclosed BSorNWS-clutch mechanical joint is a BSorNWS clutch that includes only a single wrap spring or two nested wrap springs. As stated in U.S. Pat. No. 11,020,247, it was believed by the inventor at the time of filing of U.S. Pat. No. 11,020,247 that a dual wrap spring was necessary for implementation of a mechanical-knee joint:
a. A single wrap spring could, upon mechanical failure, lead to accidents. By using two wrap springs, a single-spring failure does not result in failure of rotational constraint, but is noticeable to an amputee, alerting the amputee that repair is needed. Furthermore, the dual-wrap-spring configuration provides symmetrical distribution of loading forces through the two-way, by-passable, overrunning mechanical clutch, relieving unbalanced stress and potential failure modes.

However, further research and development efforts have revealed that, in fact, the currently disclosed single-spring wrap-spring clutch (“SSWSC”) and nested-spring wrap-spring clutch (“NSWSC”) are reliable and have many important advantages over the previously disclosed WSC. First, both SSWSCs and NSWSCs are more compact and are therefore easier to incorporate into a prosthetic leg. The increased compactness, or decreased volume, of the SSWSC and NSWSC with respect to the WSC greatly facilitates hermetically sealing a BSorNWS-clutch mechanical joint. SSWSCs and NSWSCs provide greater robustness and reliability as well as lower production costs and greater design flexibility with respect to the WSC. The single spring of an SSWSC is less expensive and easier to manufacture than the dual wrap spring of the WSC. Furthermore, it is easier to align and adjust a BSorNWS-clutch than it is to align and adjust a WSC, with dual springs that interface to two different cylindrical surfaces, namely the cylindrical surfaces of the two arbors 804 and 806. Not only does the greater length of the dual wrap spring increase the width of the WSC compared to that of a BSorNWS clutch, the greater length also decreases alignment tolerances of the dual wrap spring within the WSC assembly and increases the number of different components that must be aligned and precisely manufactured for effective operation of the WSC. While the WSC and the SSWSC both operate by selectively generating friction to inhibit rotation of the lower-leg fixture with respect to and upper-leg fixture, the increased friction associated with the two springs of the dual wrap spring used in the WSC may result in unwanted frictional drag during transitions between activation and deactivation of the clutch and friction generated on two different surfaces by two different springs is prone to asymmetric braking forces, to uneven wear, more difficult alignment, and other problems. As further discussed below, by selecting suitable materials and designs for an SSWSC, a single spring has been found to be more reliable and more robust than the dual wrap spring of the WSC.

Another significant difference between the inverted SSWS that is used in one implementation of the currently disclosed BSorNWS clutch and the dual wrap spring used in the WSC is that, as shown in FIGS. 5 and 6, the inverted single-spring wrap spring expands outward to press against the inner friction surface 408 of the cylindrical member 406 (see FIGS. 4A-B) to generate friction that inhibits rotation while, by contrast, the two springs of the WSC clamp down against the cylindrical surfaces of two arbors 804 and 806. Thus, an inverted SSWSC is activated by expansion or unwinding of the spring while the WSC is activated by contraction or increased winding of the two dual springs.

The WSC mechanical joint includes a yoke 816 in the fairly complex yoke assembly that additionally includes the dual wrap spring, two arbors, two arbor sleeves 808 and 810, two clutch pins 812 and 814, the cylindrical cam 878, a torque-transmission pin 820, two flexible linkages 224 and 226, and additional components. By contrast, the inverted SSWSC implementation of the BSorNWS-clutch mechanical joint includes a housing, cylindrical member, lower-leg and upper-leg attachment shafts, single wrap spring, and a bypass linkage, which are different from the components of the WSC mechanical joint and operate differently from operation of the components of the WSC mechanical joint, as further discussed below. Thus, a BSorNWS clutch is different from, and operates differently than, the WSC and the BSorNWS-clutch mechanical joint is different from, and operates differently than, the WSC mechanical joint disclosed in U.S. Pat. No. 11,020,247. FIG. 8 includes numerous numeric labels not mention in the preceding paragraphs but which are discussed in U.S. Pat. No. 11,020,247, which includes current FIG. 8 as FIG. 6.

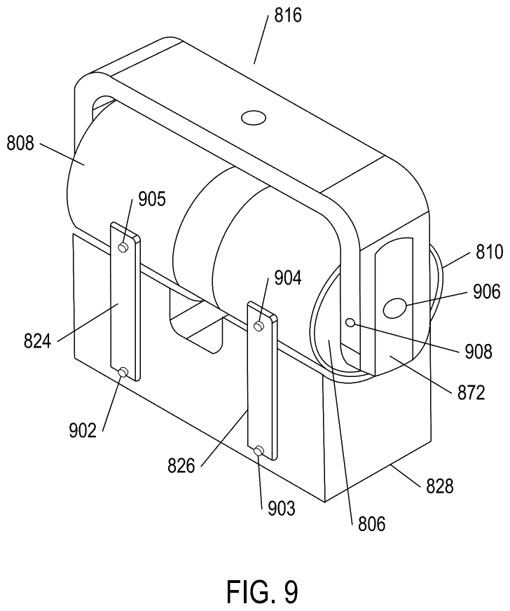

FIG. 9 shows the WSC mechanical joint from a perspective view. The WSC mechanical joint comprises the yoke assembly, including the yoke 816, arbor sleeves 808 and 810, and internal components discussed above as well as a lower-leg block 828 that translates in the vertical direction with respect to the yoke assembly depending on whether or not the lower leg of a prosthetic is weighted or unweighted. Two flexible linkages 824 and 826 link the lower-leg block 828 to arbor sleeves 808 and 806. When the lower-leg block is translated downward with respect to the yoke assembly, due to unweighting of the lower leg and lower-leg block, the arbor sleeves are rotated in a counterclockwise direction, from the perspective of FIG. 9, which unwinds the two helical coils within the yoke assembly and releases or deactivates the WSC, allowing free rotation of the lower-leg block with respect to the yoke assembly. By contrast, in the BSorNWS-clutch mechanical joint, as shown in FIGS. 5-6, lower-leg attachment shaft 422 is slidably mounted within the lower-leg-attachment-shaft fitting 414 of the cylindrical housing 410, and remains securely mounted within the lower-leg-attachment-shaft fitting whether or not the lower leg of the prosthetic leg that incorporates the BSorNWS-clutch mechanical joint is weighted or not weighted. For this reason, the BSorNWS-clutch mechanical joint is more physically secure, during the gait cycle, than the WSC mechanical joint, in which the two major parts, the yoke assembly and lower-leg block, are separate and separate from one another during unweighting of the WSC mechanical joint and the lower leg of a prosthetic leg incorporating the WSC mechanical joint. This is yet another improvement and advantage of the currently disclosed BSorNWS-clutch mechanical joint with respect to the WSC mechanical joint. Numerous additional advantages and improvements incorporated into the currently disclosed BSorNWS-clutch mechanical joint are discussed, in detail, in subsequent sections of this document.

### Currently Disclosed Methods and Systems

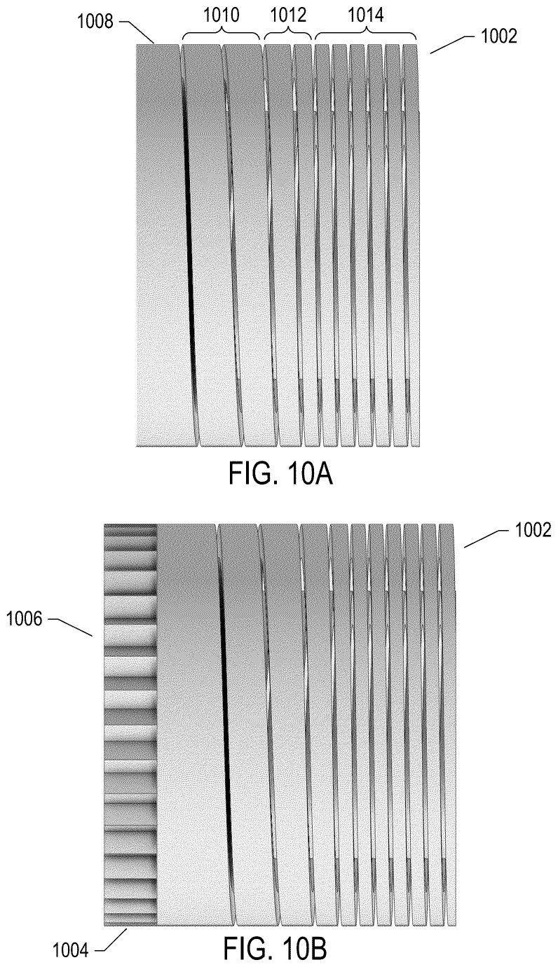

FIGS. 10A-B show one implementation of an inverted single-spring wrap spring (“inverted SSWS”) used in the currently disclosed BSorNWS clutch. FIG. 10A shows the inverted SSWS 1002 viewed from the side. FIG. 10B shows the inverted SSWS 1002 attached to a circular base plate 1004, with sprocket-like features, including sprocket feature 1006, along with circumference of the circular base plate that mate with complementary features on the interior surface of the housing, discussed below, to securely fix the base plate and left end of the inverted SSWS to the housing. This is but one example of many different types of features and mechanisms that can be used to securely rotationally fix one end of the inverted SSWS to the housing. Note that the coils of the SSWS are cut from a metal cylinder, and thus have approximately rectangular cross sections, presenting a locally flat surface to the local flat friction surface against which they are pushed when the BSorNWS clutch is activated.

The currently disclosed inverted SSWS incorporates a significant improvement with respect to the dual wrap spring used in the previously disclosed WSC. The widths of the coils vary along the length of the inverted SSWS. At the left end of the inverted SSWS, first coil 1008 has the greatest width. The widths of the next two coils 1010 are shorter than that of the first coil, but are still reasonably wide. Several even narrower coils 1012 next occur along the inverted SSWS, followed by 6 narrowest coils 1014. The narrowest coils are referred to as “teaser coils.” The friction generated between the outer, flat surface of a wrap-spring coil and the inner friction surface 408 of the cylindrical member 406 of the upper-leg component 402 (see FIGS. 4A-B) is proportional to the surface area of the outer, flat surface of the wrap-spring coil. However, the flexibility of a wrap-spring coil is inversely proportional to the width of the wrap-spring coil. The teaser coils are therefore significantly more flexible than the wider coils at the left side of the inverted SSWS but generate less friction when they conform to the inner surface of the cylindrical member than the wider coils at the left side of the inverted SSWS. When the BSorNWS clutch is activated by unwinding the inverted SSWS so that the outer surfaces of the coils are pushed against the inner friction surface of the cylindrical member, the teaser coils, being more flexible, more readily conform to the inner surface of the cylindrical member and first begin to generate fiction. The remaining coils then cooperatively engage with the inner friction surface of the cylindrical member and, once engaged, generate the frictional forces needed to inhibit rotation of the lower-leg attachment shaft with respect to the upper-leg attachment shaft in the flexion direction. The teaser coils therefore provide a relatively quicker engagement and disengagement, but are associated with less frictional drag and thus the variable-width coils together provide for rapid clutch activation and deactivation while minimizing frictional drag within the BSorNWS-clutch mechanical joint. Not only does the width of the coils vary along the length of the improved inverted SSWS, the pitch of the coils also varies along the length of the inverted SSWS. If each coil is considered to be an approximation of the cylindrical edge of a planar disk, the pitch of a coil is the angle between a vector perpendicular to the plane of the disk and the rotational symmetry axis of the inverted SSWS. By this definition, the pitch of the narrowest coils 1014 is approximately 0° while the pitch of the second-from-left coil appears to be around 5°. Thus, the improved inverted SSWS is a variable-coil-width and variable-coil-pitch inverted SSWS that provides quick engagement and disengagement of the inverted SSWS during activation and deactivation of the BSorNWS clutch while minimizing frictional drag.

The inverted SSWS needs to be made from a metal alloy that is not susceptible to residual stresses and thus will not change in shape and/or dimension during the process in which a metal-alloy cylinder is helically cut to produce the inverted SSWS. In one embodiment, the wrap string is made from 4340 high tensile steel that has been heat-treated to maximize the tensile strength.

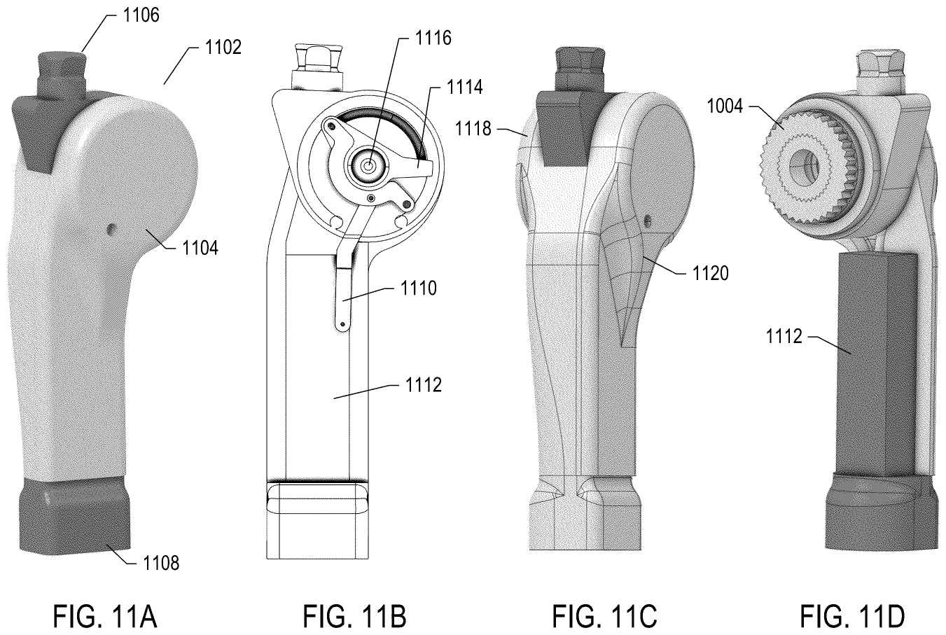

FIGS. 11A-D illustrate one implementation of the currently disclosed BSorNWS-clutch mechanical joint that is incorporated into a BSorNWS-clutch prosthetic leg. FIG. 11A shows a perspective view of the BSorNWS-clutch mechanical joint. The BSorNWS-clutch mechanical joint 1102 includes a combined housing and lower-leg-attachment-shaft fitting 1104, with the upper portion of the combined housing and lower-leg-attachment-shaft fitting equivalent to the cylindrical housing 410 of the simple, conceptual BSorNWS-clutch mechanical joint shown in FIGS. 4A-B and the lower portion of the combined housing and lower-leg-attachment-shaft fitting equivalent to lower-leg-attachment-shaft fitting 414 of the simple, conceptual BSorNWS-clutch mechanical joint shown in FIGS. 4A-B. A standard inverted-pyramid-shaped feature 1106 corresponds to the upper-leg attachment shaft 404 of the simple, conceptual BSorNWS-clutch mechanical joint shown in FIGS. 4A-B and a tube clamp 1108 at the end of a shaft corresponds to the lower-leg attachment shaft 422 of the simple, conceptual BSorNWS-clutch mechanical joint shown in FIGS. 4A-B. The wrap-spring clutch is, of course, contained within the upper portion of the combined housing and lower-leg-attachment-shaft fitting. The implementation shown in FIG. 11A is but one of many possible implementations using different types of lower-leg and upper-leg attachment features, which can include screw threads and complementary threaded features, various types of compression-fit features, and other types of attachment features. In the remainder of this document, the various different types of upper-leg shafts and/or attachment features will be referred to as the “upper-leg attachment” and the various different types of lower-leg shafts and/or attachment features will be referred to as the “lower-leg attachment.” The combined housing and lower-leg-attachment-shaft fitting will be referred to as the “housing.” As can be appreciated by review of FIG. 11A, the currently disclosed BSorNWS-clutch mechanical joint is a practical and implementable mechanical joint that can be fully sealed and incorporated into a prosthetic leg, unlike the simple, conceptual BSorNWS-clutch mechanical joint shown in FIGS. 4A-B, but the currently disclosed BSorNWS-clutch mechanical joint operates in a fashion similar to that of the above-discussed simple, conceptual BSorNWS-clutch mechanical joint shown in FIGS. 4A-B.

FIG. 11B shows the implementation of the currently disclosed BSorNWS-clutch mechanical joint shown in FIG. 11A with a portion of the housing cutaway to reveal the bypass mechanism that operates in similar fashion to the bypass linkage of the simple, conceptual BSorNWS-clutch mechanical joint shown in FIGS. 4A-B. The bypass mechanism includes an attachment arm 1110 that is mounted to the shaft of the lower-leg attachment 1112 and an attachment bracket 1114 that is attached to the end of the inverted SSWS closest to the viewer in the perspective shown in FIG. 11B. In the following discussion, this end of the inverted SSWS is referred to as the “lower-leg end” of the inverted SSWS. When the shaft of the lower-leg attachment 112 slides downward due to unweighting of the prosthetic leg in which the BSorNWS-clutch mechanical joint is incorporated, the attachment shaft of the bypass linkage is translated downward, which causes the bypass-linkage attachment bracket 1114 to rotate in a clockwise direction, in the perspective of FIG. 11B, about a rotation axis coincident with a cylindrical shaft, the end 1116 of which can be seen in FIG. 11B. Clockwise rotation of the inverted SSWS tightens the coils and releases the coils from contact with the interior friction surface of the cylindrical member within the housing, deactivating the BSorNWS clutch. When the shaft of the lower-leg attachment 112 slides upward due to weighting of the prosthetic leg in which the BSorNWS-clutch mechanical joint is incorporated, the attachment shaft of the bypass linkage is translated upward, which causes the bypass-linkage attachment bracket 1114 to rotate in a counterclockwise direction, in the perspective of FIG. 11B. Counterclockwise rotation of the inverted SSWS releases the coils to expand against the interior friction surface of the cylindrical member within the housing, activating the BSorNWS clutch and preventing further rotation of the lower-leg attachment with respect to the upper leg-attachment in the counterclockwise direction, or lower-leg flexion direction.

FIG. 11C shows the implementation of the currently disclosed BSorNWS-clutch mechanical joint shown in FIG. 11A slightly rotated about a vertical axis to reveal a portion of the side of the upper housing 1118 not visible in FIG. 11A, with curved lines, such as curved line 1120, indicating the shape of the housing. FIG. 11D shows the implementation of the currently disclosed BSorNWS-clutch mechanical joint shown in FIG. 11C with the housing removed to reveal the shaft of the lower-leg attachment 112 and the base plate 1004 of the inverted SSWS. As mentioned above, the sprocket-like features of the exterior edge of the base plate mate with complementary features in the housing to rotationally fix the housing end of the inverted SSWS, closest to the viewer in the perspective shown in FIG. 11D, to the housing. In other words, the housing end of the inverted SSWS is coupled to the housing while the lower-leg end of the housing is attached, through the bypass mechanism, to the shaft of the lower-leg attachment. As with the simple, conceptual BSorNWS-clutch mechanical joint shown in FIGS. 4A-B, the upper-leg attachment 1106 is rotationally fixed to the cylindrical member within the housing. When the BSorNWS clutch is activated, the cylindrical member and upper-leg attachment cannot rotate with respect to the lower-leg attachment in the flexion direction. BSorNWS clutch is deactivated, the cylindrical member and upper-leg attachment can freely rotate with respect to the lower-leg attachment in both the flexion direction and the extension direction.

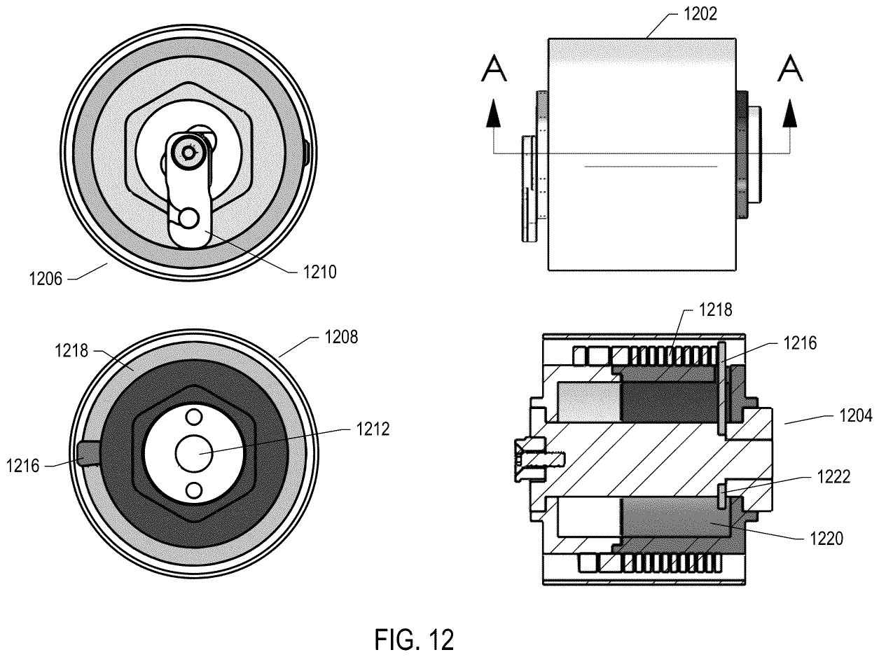

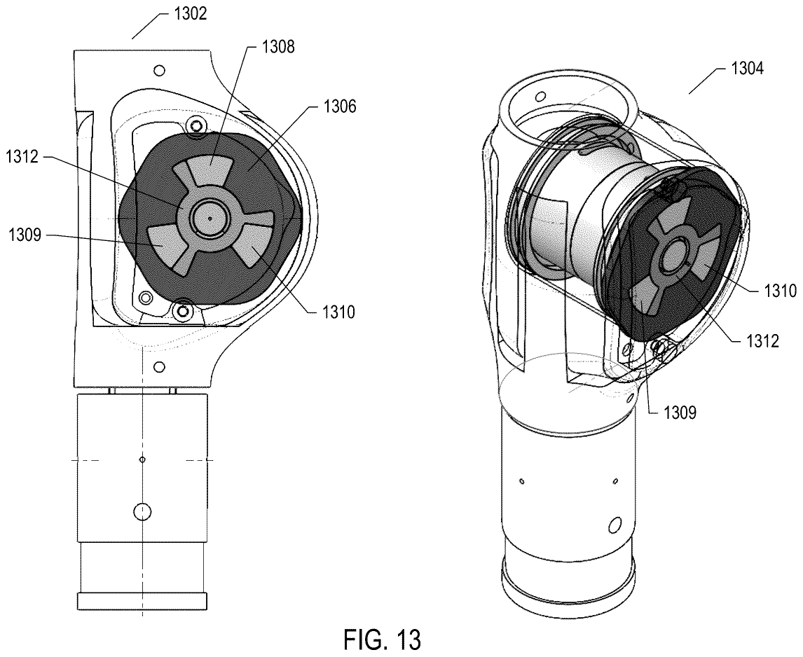

There are many different possible alternative bypass mechanisms that can be included in alternative implementations of the BSorNWS-clutch mechanical joint. As one example, an attachment bracket may be connected via a flexible linkage, such as by a cord or wire, to the lower-leg-attachment shaft rather than by an attachment arm, in the implementation shown in FIG. 11B. The flexible linkage may be attached, via a mechanical arm, to the cylindrical shaft near the housing and of the inverted SSWS, rather than to the attachment bracket. FIG. 12 illustrates this alternative bypass mechanism. A perspective side view of the cylindrical member is shown in the center of FIG. 12. A section 1204 through the side view is shown in the lower right-hand side of FIG. 12. End-on views 1206 and 1208 of the cylindrical member and mechanisms within the cylindrical member are shown on either side of the perspective side view. The mechanical arm 1210 is attached to one end of the cylindrical shaft, the other end 1212 of which is shown in end-on view 1208. A small attachment bracket 1216 attached is the cylindrical cylinder to the end of the inverted SSWS 1218. Section 1204 shows the attachment bracket 1216 attached to the end of the inverted SSWS 1218. In this embodiment, there may be a second, nested inverted SSWS 1222 to which another attachment bracket 1222 is attached. Nested-wrap-spring limitations are discussed, further, below. a. As mentioned above, in a preceding subsection of this document, a mechanical knee joint should prevent substantial flexion of the lower leg with respect to the upper leg at positions or time points 112 and 114 in the gait cycle illustrated in FIG. 1A, allow small amount of controlled flexion at positions or time points 111 and 113 in the gait cycle illustrated in FIG. 1A, and allow substantial flexion at positions or time points 115-117 in the gait cycle illustrated in FIG. 1A. FIG. 13 illustrates one implementation of a flexion-prevention-override mechanism that allows a small amount of controlled flexion of the BSorNWS-clutch mechanical joint at positions or time points 111 and 113 in the gait cycle illustrated in FIG. 1A. FIG. 13 provides a side view 1302 and a perspective 1304 of the flexion-prevention-override mechanism within the BSorNWS-clutch mechanical joint. This mechanism includes a slotted ring 1306 affixed to the cylindrical member and affixed to the lower-leg end of the inverted SSWS 1308-1310, and a compressible urethane spider 1312. As the lower-leg attachment rotates in the flexion direction with respect to the upper-leg attachment and cylindrical member when the BSorNWS clutch is activated, the faces of the lugs in contact with faces of the spider arms are forced into the spider arms, compressing the urethane and providing a small angular rotation of the lower-leg attachment in the flexion direction with respect to the upper-leg attachment and cylindrical member even though the inverted SSWS has expanded against the inner surface of the cylindrical member to prevent rotation of the cylindrical member with respect to the inverted SSWS. The small angular relaxation is the small amount of controlled flexion, at positions or time points 111 and 113 in the gait cycle illustrated in FIG. 1A, permitted by the BSorNWS-clutch mechanical joint. The angular extent of the small angular relaxation can be controlled by using different spiders of different compressibility. There are various alternative implementations of the flexion-prevention-override mechanism, as further discussed below. The flexion-prevention-override mechanism was not provided by the previously disclosed WSC mechanical joint and thus represents yet another improvement incorporated into the currently disclosed BSorNWS-clutch mechanical joint with respect to the previously disclosed WSC mechanical joint.

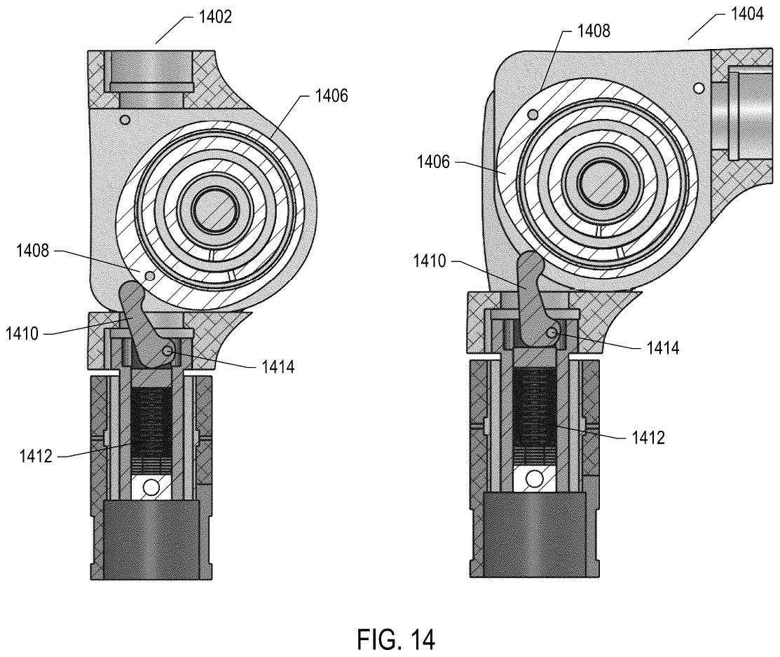

FIG. 14 illustrates yet another improvement incorporated into the currently disclosed BSorNWS-clutch mechanical joint. When the BSorNWS-clutch mechanical joint is used as a mechanical knee in a prosthetic leg, the threshold force generated by weighting of the lower leg needed to activate the BSorNWS needs to vary depending on the rotational orientation of the lower-leg attachment of the upper-leg attachment. When a user of the prosthetic leg is standing on the prosthetic leg, at full extension of the lower leg with respect to the upper leg, as depicted in cross-section 1402, the prosthetic leg is fully weighted. By contrast, when the user is sitting and the lower-leg attachment is an angle of 90° with respect to the upper-leg attachment, as depicted in class-section 1404, the prosthetic leg is only slightly weighted, since the majority of the user's weight is transferred to the chair or other object in which the user is sitting. When the user begins to stand up from a sitting position, the BSorNWS clutch needs to be activated, to prevent the prosthetic leg from buckling, but, as mentioned above, only a small amount of weight is being transferred to the lower leg of the prosthetic leg when the user is in this position. By contrast, while the BSorNWS clutch also needs to be activated in the standing position, as depicted in cross-section 1402, the prosthetic leg is fully weighted, so that any weighting less than full weighting could be used as a threshold weighting for BSorNWS-clutch activation in the standing position. In fact, it is desirable that the threshold weighting for activating the BSorNWS clutch be relatively large, at or near full extension, so that the BSorNWS clutch is deactivated at the appropriate position and time point in the gait cycle. It is also desirable that the threshold weighting for activating the BSorNWS clutch be relatively small, at 90° flexion, so that the BSorNWS clutch is activated as a user begins to stand from a sitting position.

FIG. 14 shows one implementation of a mechanism for varying the activation weighting threshold. The mechanism includes an asymmetrical cylinder 1406 with a cam-like protrusion 1408 that is rotationally fixed to the upper-leg attachment, a cam follower 1410, and a spring 1412. At 0° flexion, as shown in cross section 1402, in the standing position, the cam rotates the cam follower outward about hinge 1414, depressing the spring 1412. The spring then produces a force in a direction opposite from the force produced by the weighted lower leg, increasing the threshold weighting for activating the BSorNWS clutch. By contrast, at 90° flexion, as shown in cross-section 1404, in the sitting position, the cam follower is in a more upright position and is thus not pushing down on the spring. Because the spring is relatively decompressed, it generates a much smaller force, or no force, in opposition to the force generated by weighting of the lower leg. Therefore, the threshold weighting for activating the BSorNWS clutch is much lower.

In summary, the currently disclosed BSorNWS-clutch mechanical joint comprises the following main components: (1) a housing; (2) a BSorNWS clutch enclosed within the housing; (3) a lower-leg attachment that translates towards the housing when the BSorNWS-clutch mechanical joint is weighted and away from the housing and when the BSorNWS-clutch mechanical joint is unweighted; (4) an upper-leg attachment, the lower-leg attachment rotating with respect to the upper-leg attachment in an extension direction up to full extension, but prevented from rotating in a flexion direction when the BSorNWS clutch is activated; and (5) a mechanical bypass that activates the bypassable wrap-spring clutch when the lower-leg attachment translates towards the housing and that and deactivates the BSorNWS clutch when the lower-leg attachment translates away from the housing. Certain implementations of the currently disclosed BSorNWS-clutch mechanical joint additionally include: (6) a position-dependent weighting-threshold adjuster; and (7) a mechanical flexion-prevention override that allows a small amount of rotation of the lower-leg attachment with respect to the upper-leg attachment in the flexion direction when the BSorNWS-clutch mechanical joint is weighted. Each of the 5 main components of the currently disclosed BSorNWS-clutch mechanical joint and the two additional components of the currently disclosed BSorNWS-clutch mechanical joint can be implemented in numerous different, alternative ways, some of which are discussed in the following subsection of this document.

### Various Alternative Implementations of the Currently Disclosed BSorNWS-Clutch-Incorporating Mechanical Joint

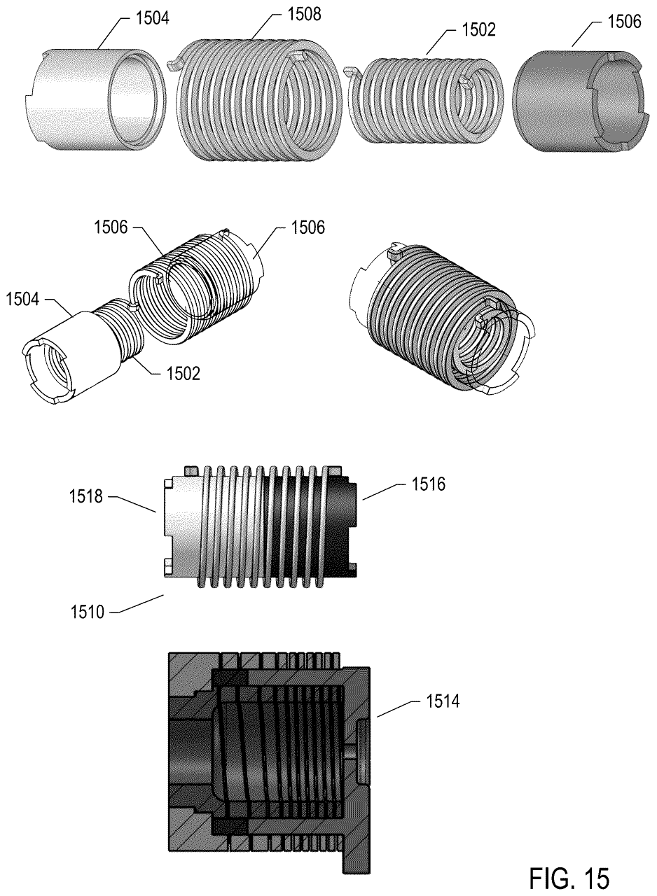

FIG. 15 illustrates a nested-spring wrap spring (“NSWS”) and a BSorNWS clutch containing an NSWS. The NSWS illustrated in FIG. 15 includes: (1) a smaller-diameter inner inverted single-spring wrap spring (“SSWS”) 1502 with coils wrapped in a first direction; (2) a two-piece cylindrical member 1504 and 1506; and (3) a larger-diameter outer SSWS 1508 wrapped in a second direction. The inner inverted SSWS 1502 is positioned within the two-part cylindrical member 1504 and 1506. The two-part cylindrical member containing the inner inverted SSWS is then positioned within the outer SSWS 1508. FIG. 15 shows a perspective view 1510 of the assembled NSWS. Finally, FIG. 15 shows an assembled NSWS clutch 1514. An NSWS clutch is activated when the inner inverted SSWS expands outward to press against the inner surface of the two-part cylindrical member and the outer SSWS clamps down onto the outer surface of the two-part cylindrical member. Viewing the assembled NSWS 1510 down the long axis of symmetry from end 1516, an NSWS containing the assembled NSWS 1510 is activated when the proximal end of the inner and outer SSWSs, attached to the housing near the distal and 1518, are rotated in a clockwise direction with respect to the two-part cylindrical member and is deactivated when the inner and outer SSWSs are rotated in a counterclockwise direction. An NSWS and a BSorNWS clutch containing the NSWS, like an SSWS and a BSorNWS clutch containing the SSWS, is more compact than a previously disclosed dual wrap spring and WSC. Thus, as mentioned above, a BSorNWS clutch, whether using an SSWS or an NSWS, can be used to implement a BSorNWS-clutch mechanical joint that is more easily hermetically sealed and which offers for more design flexibility for use as a mechanical knee in a prosthetic leg. A BSorNWS clutch can best be implemented by using: (1) an SSWS that clamps down onto a cylindrical frictional surface to activate the clutch; (2) an inverted SSWS that expands outward to press against a cylindrical frictional surface to activate the clutch; and (3) an NSWS, such as NSWS 1510 shown in FIG. 15.

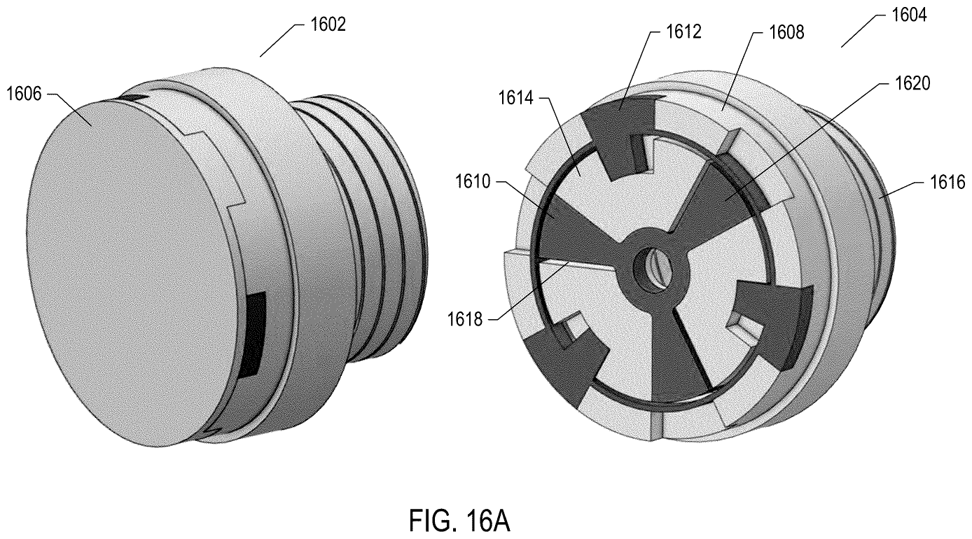

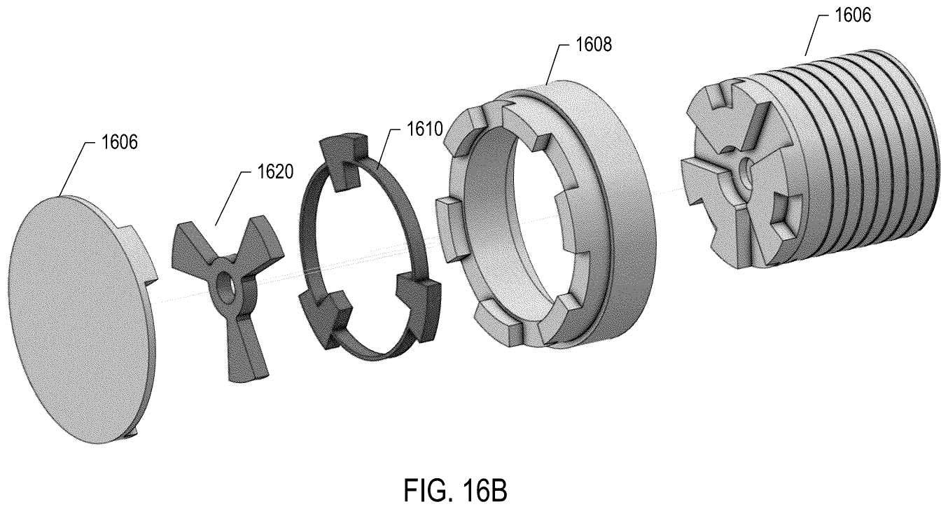

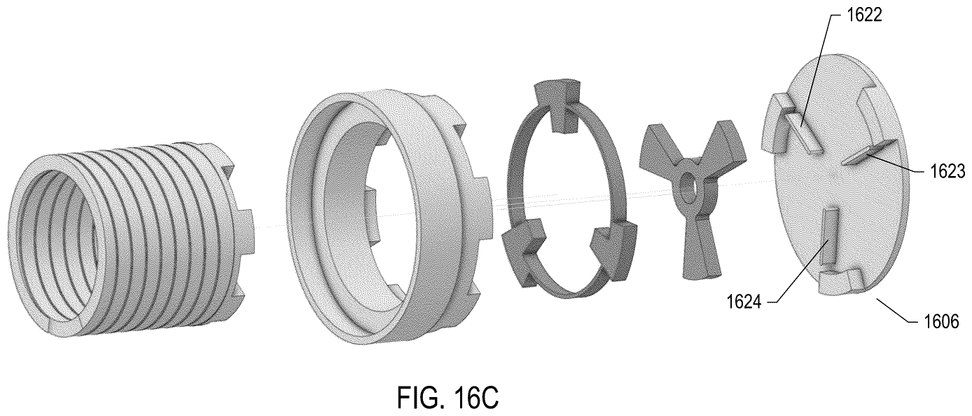

FIGS. 16A-C illustrate an alternative flexion-prevention-override mechanism that allows a small amount of rotation of the lower-leg attachment with respect to the upper-leg attachment in the flexion direction when the BSorNWS-clutch mechanical joint is weighted. FIG. 16A shows a perspective view 1602 of the upper portion of the housing (1118 in FIG. 11C) in which the alternative flexion-prevention-override mechanism is incorporated. A second perspective view 1604, shows the upper portion of the housing with cap 1606 removed. The mechanism includes a first slotted ring 1608 with slots and tabs complementary to slots and tabs in cap 1606. Ring 1610 includes stop features, such as stop feature 1612, outer portions of which fit within slots in the first ring. Larger, two-prong lugs, such as lug 1614, are affixed to the housing end of an inverted SSWS 1616. A urethane spider 1620 fills the spaces between the lugs. The spider is rotationally fixed to the housing via spider tabs on the inside surface of 1606 that insert into spaces between the spider and lugs, such as space 1618. When the BSorNWS clutch is activated, the housing, rotationally fixed to the lower-leg attachment, can rotate a bit further, in the counterclockwise direction from the perspective of FIG. 16A, until the surfaces of the leading edges of the lugs rotate against the surfaces of the trailing edges of the stop features and they rotate a bit further still by compressing the urethane spider. By replacing ring 1610 with a different ring having stop features with wider or narrower prongs, the amount of flexion-prevention override provided by the flexion-prevention-override mechanism can be changed. FIG. 16B shows an exploded diagram of the flexion-prevention-override mechanism discussed above with reference to FIG. 16A. FIG. 16C shows the exploded diagram from a different perspective than that of FIG. 16B. Note that the spider tabs 1622-1624 on the inner side of 1606 are visible in this perspective.

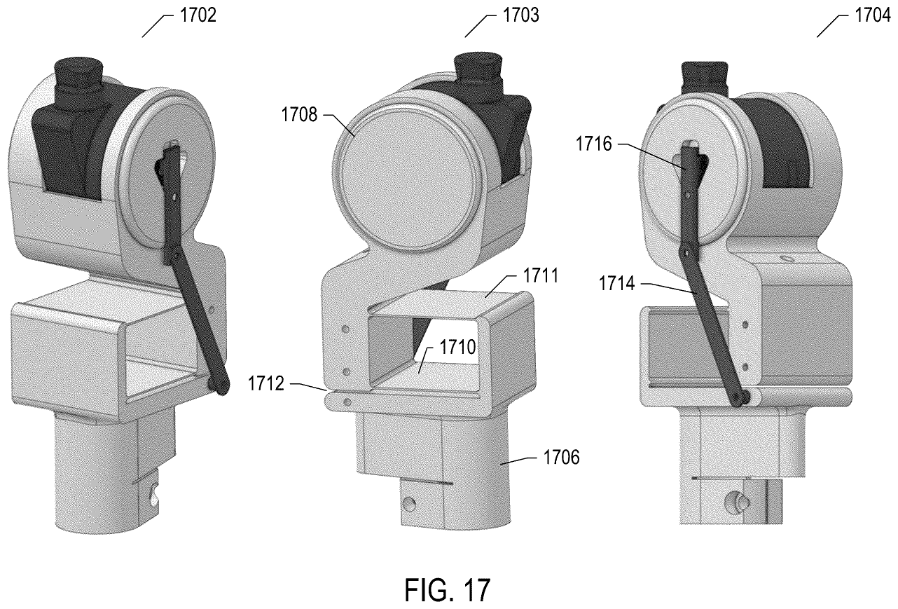

FIG. 17 shows an alternative implementation of the lower-leg attachment that, as discussed above, translates with respect to the housing depending on whether or not the lower-leg attachment is weighted or unweighted. In the alternative implementation, three perspective views 1702-1704 of which are shown in FIG. 17, the lower-leg attachment 1706 is attached to the housing 1708 via a pair of thin, planar flexures 1710-1711. The lower-leg attachment needs to translate with respect to the housing by only a few millimeters, which is represented by the narrow gap 1712 in the implementation shown in FIG. 17. Weighting of the lower-leg attachment closes this gap while unweighting of the lower-leg attachment opens the gap by one or more millimeters, and this small translation is communicated through the bypass linkage 1714 to the BSorNWS clutch. In the alternative implementation, the bypass mechanism is activated by downward translation of the upper arm 1716 of the bypass linkage which is transduced into a rotation of one end of the SSWC, inverted SSWC, or NSWC within the BSorNWS clutch.

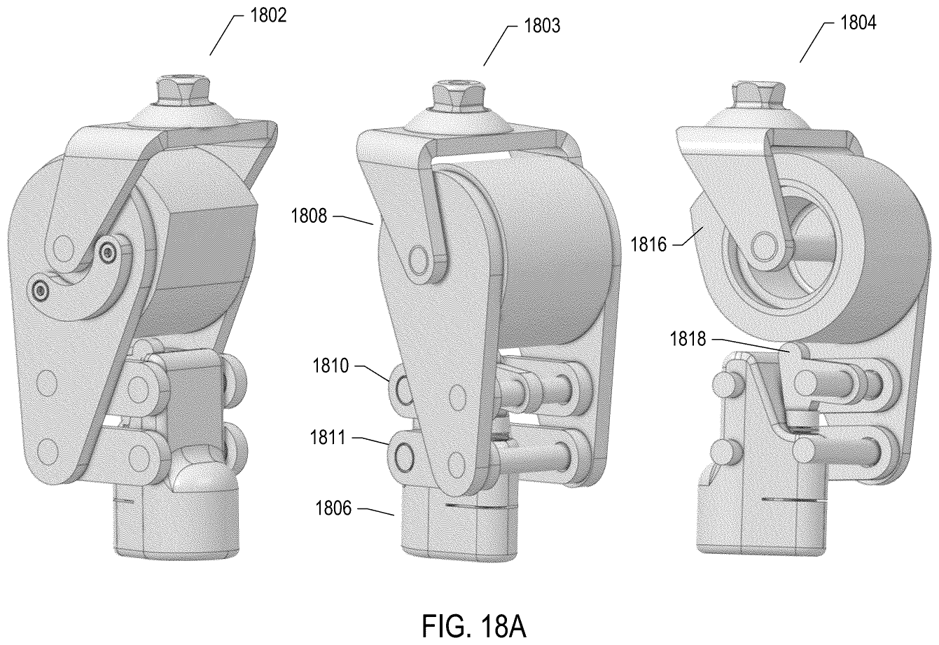

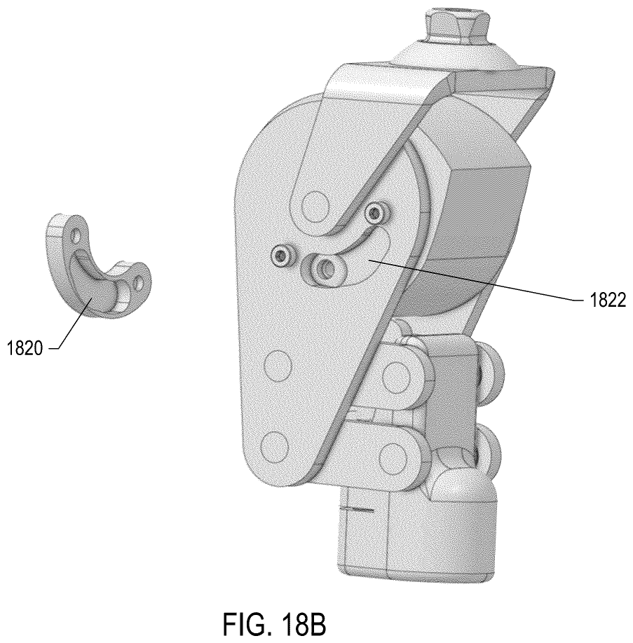

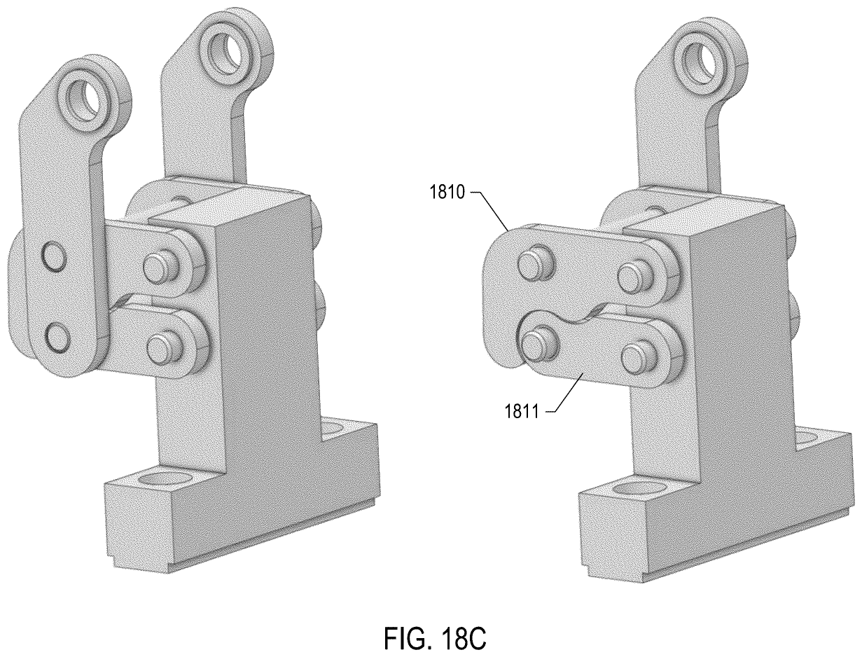

FIGS. 18A-C illustrate yet a different, alternative implementation of the lower-leg attachment. FIG. 18A shows three perspective views 1802-1804 of this implementation. The lower-leg attachment 1806 is attached to an upper portion of the BSorNWS-clutch mechanical joint 1808 via a pair of rotating, approximately horizontal arms 1810-1811. Small rotations of these arms result in vertical translation of the lower-leg attachment 1806 with respect to the upper portion of the BSorNWS-clutch mechanical joint. The previously discussed mechanism for varying the activation weighting threshold can be seen in the cam-like shape of housing 1816 and the cam follower 1818. As shown in FIG. 18B, a flexion-prevention-override mechanism that includes cap 1820 and recess 1822, in which a urethane compression feature resides, is included in the implementation shown in FIGS. 18A-C. As shown in FIG. 18C, the pair of rotating, approximately horizontal arms 1810-1811 lock together to prevent more than a small vertical translation of the lower-leg attachment with respect to the upper portion of the BSorNWS-clutch mechanical joint when the lower-leg attachment is unweighted.

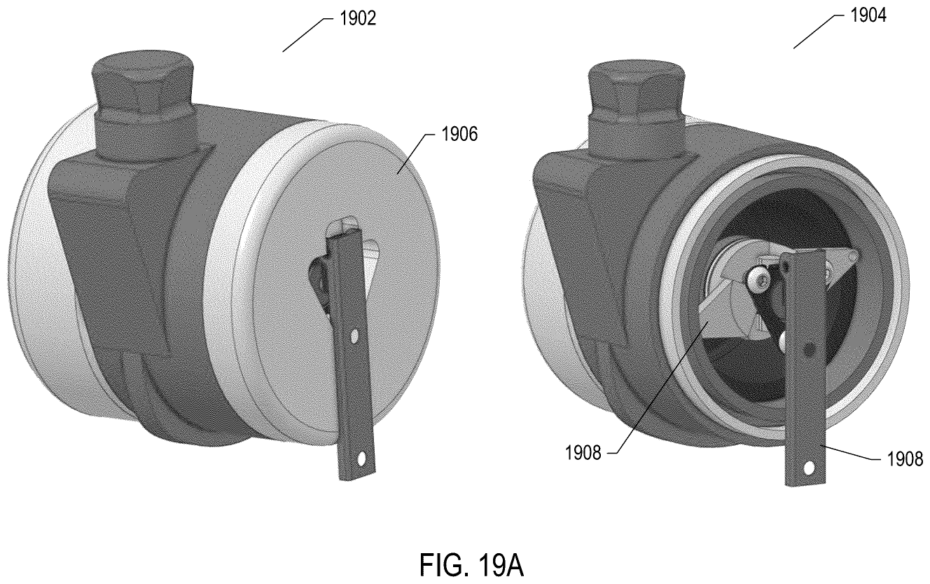

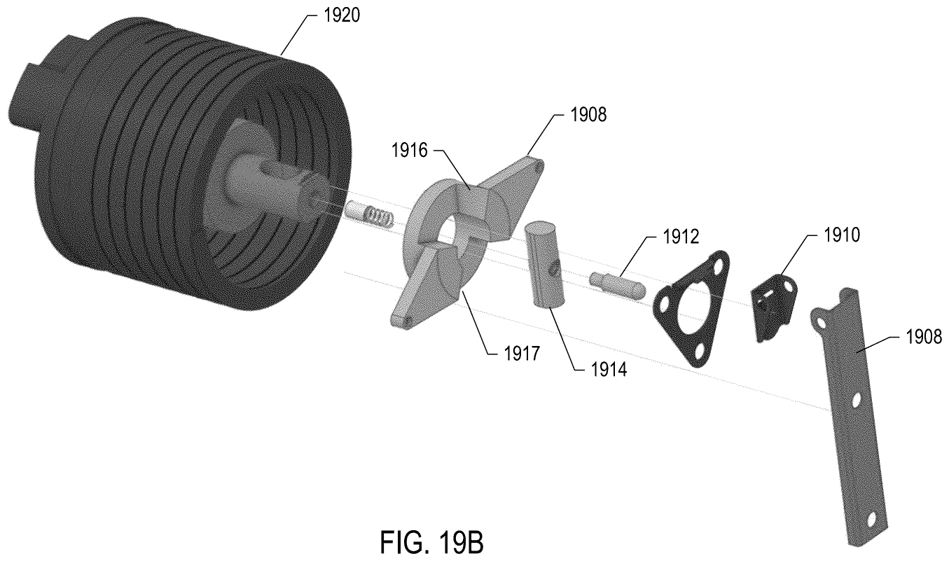

FIGS. 19A-B illustrate the alternative bypass mechanism mentioned above with reference to FIG. 17. FIG. 19A shows two perspective views 1902 and 1904 of the alternative bypass mechanism, with view 1904 showing assembled internal components with the cap-like portion 1906 of the housing removed. The upper arm 1908 of the bypass linkage moves vertically up and down when the lower-leg attachment is weighted and unweighted, respectively. This vertical translation is transduced into a rotational motion of the SSWC, inverted SSWC, or NSWC within the BSorNWS clutch imparted by rotational motion of a winged bracket 1908. FIG. 19B shows an exploded view of the alternative bypass mechanism. The upper arm of the bypass linkage 1908 rotates hinge 1910, and rotation of the hinge when the upper arm is translated downward depresses rounded shaft 1912 inward, towards the SSWC, inverted SSWC, or NSWC within the BSorNWS clutch. This pushes the vertical shaft with elliptical cross-section 1914 into the curved faces 1916 and 1917 of the winged bracket 1908 which, in turn, causes the winged bracket to impart a rotation to the end of the inverted SSWC 1920 shown in FIG. 19B. This alternative bypass mechanism has the advantage that the bypass mechanism operates correctly regardless of the angular alignment of the winged bracket 1908 to the end of the inverted SSWC 1920.

To recapitulate, the currently disclosed BSorNWS-clutch mechanical joint comprises the following main components: (1) a housing; (2) a BSorNWS clutch; (3) a lower-leg attachment; (4) an upper-leg attachment; and (5) a mechanical bypass. Certain implementations of the currently disclosed BSorNWS-clutch mechanical joint additionally include: (6) a position-dependent weighting-threshold adjuster; and (7) a mechanical flexion-prevention override. Each of the 5 main components and two additional components can be implemented in many different ways. The housing can have various shapes and dimensions and can be made from metals or metal alloys or from composite materials, such as fiberglass and various types of polymeric materials. As discussed above, an SSWC-implemented, inverse-SSWC-implemented, and NSWS-implemented BSorNWS-clutch mechanical joint occupies less volume than the previously disclosed WSC mechanical joint, and use of the BSorNWS-clutch mechanical joint allows the housing to be more compact and more easily hermetically sealed for application in prosthetic legs. Furthermore, this compactness reduces the need for complex and fine-granularity adjustments and alignment associated with the longer dual-wrap-spring clutch.

The BSorNWS clutch, as discussed above, can be implemented using an SSWC, an inverted SSWC, or an NSWS. The BSorNWS clutch, unlike the previously disclosed WSC, is fully contained within, and supported by, the housing, simplifying manufacture of the BSorNWS clutch and significantly increasing its reliability and robustness. As discussed above, the BSorNWS clutch uses one or more wrap springs with variable coil widths and variable coil pitch, which reduces undesirable clutch-activation and clutch-deactivation temporal latencies and undesirable frictional forces that do not contribute to clutch activation and clutch deactivation. Different implementations of the currently disclosed BSorNWS clutch may feature SSWCs, inverted SSWCs, and NSWSs with different numbers of coils, different coil widths and pitches, different materials used for fabricating the SSWCs, inverted SSWCs, and NSWSs, different sizes and shapes, different types of attachments to the housing and to the mechanical bypass, and may differ in other ways from one another.

The lower-leg and upper-leg attachments may include different mechanical features for attachment to prosthetic lower legs and upper legs. The lower-leg attachment may be slidably mounted within a lower-leg-attachment fitting, as in the implementation discussed above with reference to FIGS. 11A-D, but may be alternatively implemented, as discussed above with reference to FIG. 17 and FIGS. 18A-C. Many other alternative implementations are possible to allow for controlled translation of the lower-leg attachment with respect to the housing depending on whether or not the lower-leg attachment is weighted or unweighted. Various different implementations of the mechanical bypass are discussed, above, with reference to FIGS. 4A-7E, FIG. 11B, FIG. 12, and FIGS. 19A-B, but many additional implementations are possible. Similarly, many different implementations of the position-dependent weighting-threshold adjuster and flexion-prevention override mechanism are possible, including those discussed above with reference to FIG. 13, FIG. 14, and FIGS. 16A-C.

A different, alternative mechanical bypass can be implemented by using an arbor onto which an inverted SSWS clamps down. The arbor is rotated by the bypass mechanism independently of the cylindrical member that the inverted SSWS expands onto to activate the clutch. In essence, the arbor and cylindrical member together comprise nested frictional surfaces in between which the inverted SSWS is positioned. To deactivate the clutch containing the nested frictional surfaces and inverted SSWS, the arbor is rotated in a direction that causes the inverted SSWS to clamp down on the arbor, thus releasing the inverted SSWS from contact with the inner frictional surface of the cylindrical member. Similarly, to activate the clutch, the arbor needs to be rotated slightly in the opposite direction to release the coils or the inverted SSWS. In order for this alternative mechanical bypass to operate, the outer surfaces of the coils of the inverted SSWS and the inner surfaces of the coils of the inverted SSWS cannot be simultaneously pressed against the two nested frictional surfaces of the cylindrical member and arbor, during clutch activation and deactivation, since that would prevent a state transition of the clutch from activated to deactivated and deactivated to activated states. There are different ways to prevent the outer surfaces of the coils of the inverted SSWS and the inner surfaces of the coils of the inverted SSWS from being simultaneously pressed against the two nested frictional surfaces during both weighting and unweighting, including varying the diameter of the inverted SSWS along the length of the SSWS, by varying the diameter of the cylindrical member, or by extending the SSWS past the end of the cylindrical member, so that the extended portion, alone, interacts with the arbor.

In a sense, the currently disclosed BSorNWS-clutch mechanical joint is a modular system defined by the functionalities of, and interactions between, the above-listed main components and two additional compartments. While the components or modules may be differently implemented in different implementations of the currently disclosed BSorNWS-clutch mechanical joint, all of the implementations of the currently disclosed BSorNWS-clutch are commonly defined by the functionalities of, and interactions between, the modules or components.

The present invention has been described in terms of particular embodiments, it is not intended that the invention be limited to these embodiments. Modifications within the spirit of the invention will be apparent to those skilled in the art. Many examples of alternative implementations of the various components and modules of the currently disclosed BSorNWS-clutch mechanical joint have been described and illustrated in the current document. The currently disclosed BSorNWS-clutch mechanical joint, and other types of mechanical joints that employ BSorNWS clutches, may be used in many different applications in addition to prosthetics, including in robots, complex mechanical and electro-mechanical systems, and many other applications.

1. A mechanical joint comprising:
a housing;
a clutch contained within the housing, the clutch one of a single-spring wrap-spring clutch, an inverted single-spring wrap-spring clutch, and a nested-spring wrap-spring clutch;
a lower-leg attachment that that translates towards the housing when the mechanical joint is weighted and that translates away from the housing when the mechanical joint is unweighted;
an upper-leg attachment, wherein the lower-leg attachment rotates with respect to the upper-leg attachment in an extension direction up to full extension but is prevented from rotating in a flexion direction when the clutch is activated; and
a mechanical bypass that activates the clutch when the lower-leg attachment translates towards the housing and deactivates the clutch when the lower-leg attachment translates away from the housing.

2. The mechanical joint of claim 1 wherein the housing contains the clutch and the mechanical bypass and partially contains the lower-leg attachment.

3. The mechanical joint of claim 1 wherein the housing hermetically seals the clutch and the mechanical bypass.

4. The mechanical joint of claim 1 wherein the single-spring wrap-spring clutch contains a spring with coils of varying width and pitch that clamp down on a frictional surface when the clutch is activated and that release from the frictional surface when the clutch is deactivated.

5. The mechanical joint of claim 1 wherein the inverted single-spring wrap-spring clutch contains a spring with coils of varying width and pitch that expand onto a frictional surface when the clutch is activated and that release from the frictional surface when the clutch is deactivated.

6. The mechanical joint of claim 1 wherein the nested-spring wrap-spring clutch comprises:
a cylindrical member;
a first spring with coils of varying width and pitch that expand onto an inner frictional surface of the cylindrical member when the clutch is activated and that release from the inner frictional surface of the cylindrical member when the clutch is deactivated; and
a second spring with coils of varying width and pitch that clamp down onto an outer frictional surface of the cylindrical member when the clutch is activated and that release from the outer frictional surface of the cylindrical member when the clutch is deactivated.

7. The mechanical joint of claim 1 wherein the lower-leg attachment is slidably mounted partially within the housing.

8. The mechanical joint of claim 1 wherein the lower-leg attachment is mounted by one or more flexures to the housing.

9. The mechanical joint of claim 1 wherein the lower-leg attachment is mounted by one or more rotating arms to the housing.

10. The mechanical joint of claim 1 wherein the upper-leg attachment is mounted to a cylindrical member of the clutch that constrains rotational motion of the upper-leg attachment relative to the lower-leg attachment in the flexion direction when the clutch is activated.

11. The mechanical joint of claim 1 wherein the mechanical bypass transduces the translational motion of the lower-leg attachment with respect to the housing to a rotational motion imparted to one end of a single-spring wrap-spring or inverted single-spring wrap spring within the clutch.

12. The mechanical joint of claim 1 wherein the mechanical bypass transduces the translational motion of the lower-leg attachment with respect to the housing to a rotational motion imparted to adjacent ends of two springs of nested-spring wrap-spring within the clutch.

13. The mechanical joint of claim 1 wherein the mechanical bypass transduces the translational motion of the lower-leg attachment with respect to the housing to a rotational motion imparted to one end of both springs or a nested-spring wrap-spring within the clutch.

14. A mechanical joint of claim 1 further comprising a flexion-prevention override that permits rotation of the lower-leg attachment with respect to the upper-leg attachment in the flexion direction when the clutch is activated.

15. The mechanical joint of claim 14 wherein the flexion-prevention override permits between 0° and 12° of rotation in the flexion direction when the clutch is activated.

16. The mechanical joint of claim 15 wherein the amount of rotation in the flexion direction permitted by the flexion-prevention override depends on the compressibility of a polymeric-material component of the flexion-prevention override that is compressed to permit the rotation in the flexion direction.

17. The mechanical joint of claim 1 further comprising a position-dependent weighting-threshold adjuster that varies a threshold weighting for activation of the clutch.

18. The mechanical joint of claim 17 wherein the position-dependent weighting-threshold adjuster comprises:
a cam that rotates with the upper-leg attachment;
a spring that compresses when the lower-leg attachment translates towards the housing and decompresses when the lower-leg attachment translates away from the housing; and
a cam follower that compresses the spring when the lower-leg attachment and upper-leg attachment are parallel or nearly parallel and the lower-leg attachment fully extended or nearly fully extended.

19. The mechanical joint of claim 1 incorporated into a prosthetic leg.

20. The mechanical joint of claim 19 wherein an attachment feature of the lower-leg attachment attaches to a complementary attachment feature of a lower leg of the prosthetic leg and an attachment feature of the upper-leg attachment attaches to a complementary attachment feature of an upper leg of the prosthetic leg.
